(19) United States
(12) Patent Application Publication
(10) Pub. No.: US 2025/0152855 A1
(43) Pub. Date: May 15, 2025

(54) APPARATUS FOR SKIN TREATING INCLUDING DRUG RESERVOIR

(71) Applicant: Healux Co.,Ltd., Seoul (KR)

(72) Inventors: Soon Chul KWON, Anyang-si Gyeonggi-do (KR); Chang Hyun LEE, Seoul (KR); Hwan Ju JUNG, Seoul (KR)

(73) Assignee: Healux Co.,Ltd., Seoul (KR)

(21) Appl. No.: 18/944,226

(22) Filed: Nov. 12, 2024

(30) Foreign Application Priority Data
Nov. 13, 2023 (KR) 10-2023-0156064

(51) Int. Cl. A61M 5/50; A61M 5/14; A61M 5/142; A61M 5/158; A61M 5/172

(52) U.S. Cl. CPC A61M 5/5086; A61M 5/1413; A61M 5/14232; A61M 5/172; A61M 2005/1587; A61M 2205/15; A61M 2210/04

(57) ABSTRACT

The present disclosure relates to a medical skin treatment apparatus including a main body including a controller controlling an operation of the medical skin treatment apparatus and a power supply for supplying power to the medical skin treatment apparatus, a drug reservoir storing a drug to be injected into the skin of a patient, a hand piece receiving power from the power supply of the main body, controlled by the controller, injecting the drug into the skin of the patient, and improving the skin, and a drug hose coupling the drug reservoir and the hand piece to each other and conveying the drug, wherein the drug reservoir includes a drug container storing the drug and a stopper part inserted into an opening of the drug container to prevent leakage of the drug and having a duct through which the drug hose passes formed therein.

### CROSS-REFERENCE T0 RELATED APPLICATION

This application claims priority to Korean Patent Application No. 10-2023-0156064, filed on Nov. 13, 2023, the entire contents of which are herein incorporated by reference.

### BACKGROUND

### 1. Field of the Invention

The present disclosure relates to a skin treatment apparatus including a drug reservoir, and more particularly, to a skin treatment apparatus including a stopper part that prevents leakage of a drug inside a drug container and prevents the drug container from being crushed.

### 2. Discussion of Related Art

Ultrasound therapy is a method of treating skin using high frequency waves. This method is mainly used for cosmetic purposes and is mainly used for skin care treatment of the face, neck, and so on. Ultrasound therapy can increase skin elasticity by stimulating collagen fibers in the skin and prevent loss of skin elasticity due to aging. In addition, ultrasound therapy has effects of promoting the metabolic activity of skin cells, promoting the production of new skin cells, and brightening the skin tone. Methods of such ultrasound therapy are specifically divided into a planar ultrasound method and a focus ultrasound method. Among them, the focus ultrasound method has a better fat removal effect and higher use stability than the planar ultrasound method. The working principle of the focus ultrasound method is that an ultrasound energy converter is installed on a skin surface of a fat removal target area, ultrasound energy is transmitted intensively to subcutaneous fat tissue, and fat cell membranes at a site to which the ultrasound energy is concentrated are ruptured through cavitation caused again by high-energy ultrasonic vibration, thereby allowing contents of fat cells to flow out and be removed through the body's own metabolism. By melting and removing the fat tissue in the fat removal target area through such a process, the purpose of fat removal can be achieved, and at the same time, no damage is caused to normal tissue around the fat tissue.

A skin treatment apparatus using ultrasonic waves uses a drug to cool high-temperature heat generated together when ultrasonic waves are generated or to promote skin regeneration. To this end, a drug container containing a drug may also be embedded in the skin treatment apparatus. However, the conventional drug container has a problem that a drug may leak due to loose coupling between the drug container and the skin treatment apparatus. When the drug leaks from the drug container, there is a problem that the drug may permeate into the skin treatment apparatus around the drug container and cause a failure.

### SUMMARY OF THE INVENTION

A medical skin treatment apparatus according to the present disclosure includes a main body including a controller controlling an operation of the medical skin treatment apparatus and a power supply for supplying power to the medical skin treatment apparatus, a drug reservoir storing a drug to be injected into the skin of a patient, a hand piece receiving power from the power supply of the main body, controlled by the controller, injecting the drug into the skin of the patient, and improving the skin, and a drug hose coupling the drug reservoir and the hand piece to each other and conveying the drug, wherein the drug reservoir includes a drug container storing the drug and a stopper part inserted into an opening of the drug container to prevent leakage of the drug and having a duct through which the drug hose passes formed therein, the stopper part includes an outer body and an inner body having at least a portion coming into contact with an inner circumferential surface of the opening of the drug container, the outer body includes an outer wall and a stopper part closing surface having a ring shape and integrally formed with the outer wall, the outer wall has a cylindrical shape that has an open upper side and vertically extends, the stopper part closing surface includes a lower closing surface and a catching part, the lower closing surface is formed in a ring shape that is perpendicular to the outer wall, an outer circumferential surface of the lower closing surface is coupled to a lower end of the outer wall, the catching part extends in a radial direction of the lower closing surface and is formed to be larger than a radius of the outer wall to prevent the outer body from being completely inserted into the drug container, the inner body includes an inner wall, and an upper closing surface and the duct that are integrally formed with the inner wall, the inner wall has a cylindrical shape that is coupled to the lower closing surface while perpendicular thereto, has an open lower side, and vertically extends, a lower end of the inner wall is coupled to an inner circumferential surface of the lower closing surface, the upper closing surface is formed in a ring shape that is perpendicular to the inner wall, an outer circumferential surface of the upper closing surface is coupled to an upper end of the inner wall, the duct is formed to protrude downward while perpendicular to the upper closing surface and discharge the drug inside the drug container to the outside, and an upper end of the duct is coupled to an inner circumferential surface of the upper closing surface.

In the medical skin treatment apparatus according to the present disclosure, the outer wall and the inner wall each having a cylindrical shape may form concentric circles, the radius of the outer wall may be greater than a radius of the inner wall, the stopper part closing surface and the upper closing surface may form concentric circles, and a radius of the stopper part closing surface may be greater than a radius of the upper closing surface.

In the medical skin treatment apparatus according to the present disclosure, an outer circumferential surface of the duct may include a slope that is tilted relative to an axis passing through a center of the upper closing surface, the drug hose may be fitted from below to surround the duct, and in a state in which the drug container and the stopper part are coupled to each other, the drug may be blocked by at least one of the lower closing surface and the upper closing surface and discharged only through the duct, and the drug discharged through the duct may be supplied to the hand piece through the drug hose.

In the medical skin treatment apparatus according to the present disclosure, the stopper part may further include an air hole that includes an air inlet and an air outlet, is formed between the outer wall and the inner wall, passes through the stopper part closing surface, and is formed in an L-shape and an air hose that is formed with an elastic material and has one end fixed from above to the air inlet of the air hole and the other end extending a predetermined length to come into contact with at least one of a ceiling surface and a wall surface of the drug container.

In the medical skin treatment apparatus according to the present disclosure, the air inlet of the air hole may be formed to protrude in a vertical direction between the outer wall and the inner wall, and the air outlet of the air hole may be formed to pass through at least one of the lower closing surface and the catching part in a horizontal direction.

In the medical skin treatment apparatus according to the present disclosure, the outer body may have at least one stopper protrusion formed along an outer circumferential surface of the outer wall to allow the stopper part and the drug container to tightly come into close contact with each other, and the stopper protrusion may extend in a radial direction of the outer wall and prevent the drug from leaking between the outer wall and the drug container.

In the medical skin treatment apparatus according to the present disclosure, the duct may have at least one duct protrusion formed along the outer circumferential surface of the duct to allow the stopper part and the drug hose to tightly come into close contact with each other, and the duct protrusion may extend in a radial direction of the duct and prevent the drug from leaking between the duct and the drug hose.

In the medical skin treatment apparatus according to the present disclosure, a drug detector may be attached to at least one of an inner circumferential surface of the inner wall and a lower end of the upper closing surface, which are at an inside of the inner body, to check leakage of the drug.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a medical skin treatment apparatus according to one embodiment of the present disclosure;

FIG. 2 is a view illustrating a block diagram of various components that may be included in the medical skin treatment apparatus according to one embodiment of the present disclosure;

FIG. 3 is a view illustrating a cross-section of a base part according to one embodiment of the present disclosure;

FIGS. 4A and 4B are views illustrating a cross-section of the base part according to one embodiment of the present disclosure;

FIG. 5 is a view illustrating a cross-section of the base part according to one embodiment of the present disclosure;

FIG. 6 is a view for describing a hand piece according to one embodiment of the present disclosure;

FIG. 7 is a flowchart for describing an operation of the medical skin treatment apparatus according to one embodiment of the present disclosure;

FIG. 8 is a flowchart for describing an operation of the medical skin treatment apparatus according to one embodiment of the present disclosure;

FIG. 9 is a flowchart for describing an operation of the medical skin treatment apparatus according to one embodiment of the present disclosure;

FIG. 10 is a flowchart for describing an operation of the medical skin treatment apparatus according to one embodiment of the present disclosure;

FIG. 11 is a view illustrating a drug supply according to one embodiment of the present disclosure;

FIGS. 12A and 12B are views illustrating an operation of the drug supply according to one embodiment of the present disclosure;

FIGS. 13A and 13B are views for describing an operation of a rotator according to one embodiment of the present disclosure;

FIGS. 14A through 14C are views illustrating an operation of the drug supply according to one embodiment of the present disclosure;

FIGS. 15A through 15C are views illustrating an operation of the drug supply according to one embodiment of the present disclosure;

FIGS. 16A and 16B are views illustrating the base part according to one embodiment of the present disclosure;

FIGS. 17A and 17B are views for describing a predetermined function according to one embodiment of the present disclosure;

FIG. 18 is a view illustrating the drug supply according to one embodiment of the present disclosure;

FIG. 19 is a view illustrating a drug reservoir according to one embodiment of the present disclosure;

FIG. 20 is a view illustrating a stopper part according to one embodiment of the present disclosure;

FIGS. 21A and 21B show a cross-sectional view and a bottom view of the stopper part according to one embodiment of the present disclosure;

FIGS. 22A and 22B are views illustrating the drug reservoir according to one embodiment of the present disclosure;

FIG. 23 is a cross-sectional view of the stopper part according to one embodiment of the present disclosure; and

FIG. 24 is a cross-sectional view of the stopper part according to one embodiment of the present disclosure.

### DETAILED DESCRIPTION OF THE PRESENT INVENTION

Advantages and features of the embodiments disclosed herein and methods of achieving the same should become clear from embodiments described in detail below with reference to the accompanying drawings. However, the present disclosure is not limited to the embodiments disclosed below and may be implemented in various different forms. The present embodiments are only provided to make the present disclosure complete and completely inform those of ordinary skill in the art to which the present disclosure pertains of the scope of the invention.

Terms used in the present specification will be briefly described, and embodiments disclosed herein will be described in detail.

General terms that are currently widely used have been selected as terms used in the present specification in consideration of functions in the present disclosure, but the terms may be changed according to an intention or practice of those of ordinary skill in the art, the advent of new technology, and the like. Also, in some cases, some terms may have been arbitrarily selected by the applicant, and in such cases, the meanings of the terms will be described in detail in the corresponding part of the description of the invention. Therefore, the terms used in the present disclosure should be defined based on the meanings of the terms and the content throughout the present disclosure, instead of being simply defined based on the names of the terms.

In the present specification, a singular expression includes a plural expression unless the context clearly indicates singularity. Also, a plural expression includes a singular expression unless the context clearly indicates plurality.

Throughout the specification, when a certain part is described as “including” a certain component, this indicates that the certain part may further include other components instead of excluding other components unless the context clearly indicates otherwise.

Also, the term “-er/-or” or “part” used in the specification refers to a software or hardware component, and an “-er/-or” or “part” performs certain roles. However, the meaning of “-er/-or” or “part” is not limited to software or hardware. An “-er/-or” or “part” may be configured to be present in addressable storage media or configured to replay one or more processors. Therefore, as one example, an “-er/-or” or “part” may include components such as software components, object-oriented software components, class components, and task components, processes, functions, attributes, procedures, subroutines, program code segments, drivers, firmware, micro codes, circuits, data, databases, data structures, tables, arrays, or variables. The components and functions provided in “-ers/-ors” or “parts” may be combined into smaller numbers of components and “-ers/-ors” or “parts” or may be further separated into larger numbers of components and “-ers/-ors” or “parts.”

According to one embodiment of the present disclosure, an “-er/-or” or “part” may be implemented using a processor and a memory. The term “processor” should be interpreted in a wide sense to include a universal processor, a central processing unit (CPU), a microprocessor, a digital signal processor (DSP), a controller, a microcontroller, a state machine, and the like. In some environments, “processor” may also refer to an application-specific integrated circuit (ASIC), a programmable logic device (PLD), a field programmable gate array (FPGA), or the like. For example, the term “processor” may also refer to a combination of processing devices such as a combination of a DSP and a microprocessor, a combination of a plurality of microprocessors, a combination of one or more microprocessors combined with a DSP core, or a combination of other arbitrary configurations.

The term “memory” should be interpreted in a wide sense to include an arbitrary electronic component that can store electronic information. The term “memory” may also refer to various types of processor-readable media such as a random access memory (RAM), a read-only memory (ROM), a nonvolatile random access memory (NVRAM), a programmable read-only memory (PROM), an erasable-programmable read-only memory (EPROM), an electrically erasable PROM (EEPROM), a flash memory, a magnetic or optical data storage device, and registers. When a processor is able to read information from a memory and/or record information in the memory, the memory is referred to as being in an electronic communication state with the processor. A memory integrated in a processor is in an electronic communication state with the processor.

In the present specification, an actuator is a component that can provide a driving force. Examples of the actuator may include a motor, a linear motor, an electronic motor, a direct current (DC) motor, an alternating current (AC) motor, a linear actuator, an electrically driven actuator, etc., but the actuator is not limited thereto.

Hereinafter, embodiments will be described in detail with reference to the accompanying drawings to allow those of ordinary skill in the art to which the present disclosure pertains to easily carry out the embodiments. Also, parts unrelated to the description are omitted from the drawings to clearly describe the present disclosure.

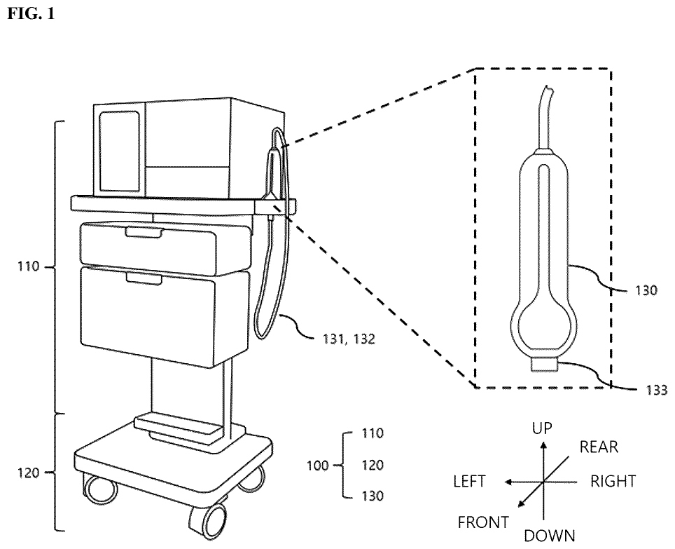

FIG. 1 is a view illustrating a medical skin treatment apparatus according to one embodiment of the present disclosure.

A medical skin treatment apparatus 100 may include a main body 110 including a controller 200 and a power supply. The main body 110 may further include at least one of a communication part, a sensor part, an output part, and an input part.

Also, the medical skin treatment apparatus 100 may include a conveyor 120 for moving the main body 110. The conveyor 120 may be located at a lower end of the main body 110. The conveyor 120 may include at least one wheel. The conveyor 120 may include at least one of a caster wheel, an electrically driven wheel, and a Mecanum wheel. The conveyor 120 may include an electric motor, and the electric motor may provide a driving force based on an input of a user. Also, the conveyor 120 may be able to autonomously navigate based on control of the controller 200. However, the present disclosure is not limited thereto, and the conveyor 120 may move by a force of the user.

Also, the medical skin treatment apparatus 100 may include a hand piece 130. The main body 110 may be coupled to the hand piece 130 through a cable 131. The hand piece 130 may be used to treat the skin of a patient. The hand piece 130 may include a handle that may be held by the user. A surface coming into contact with the skin of the patient may be formed on one side of the hand piece 130. The main body 110 may supply power to the hand piece 130 through the cable 131 and may transmit or receive a control signal via a wire.

The medical skin treatment apparatus 100 may include a drug supply 1100 coupled to a drug reservoir and supplying a predetermined amount of drug to the hand piece. The drug supply 1100 may be formed on a side surface of the main body 110 and may be attachable and detachable. The main body 110 may include the drug reservoir storing a drug to be injected into the skin of a patient. The drug reservoir may include a space storing a drug case and a cover covering the space. A user may open the cover of the drug reservoir and couple or replace a drug case to be used in treatment. A plurality of drug cases containing different drugs may be coupled to the drug reservoir. However, the present disclosure is not limited thereto, and a single drug case containing a single drug may be coupled to the drug reservoir. The drug supply 1100 may be located near the drug reservoir to rapidly supply the drug. The drug reservoir may be coupled to the drug supply 1100 through a rubber tube 132. The drug supply 1100 may be coupled to the hand piece 130 through the rubber tube 132. The rubber tube 132 may be made of rubber. However, the present disclosure is not limited thereto, and the rubber tube 132 may be made of other materials as long as the material has corrosion resistance to drugs and has a shape that can be rapidly compressed and restored. The drug supply 1100 will be described below.

The hand piece 130 may receive power from the power supply of the main body 110. The hand piece 130 may be controlled by the controller 200. The hand piece 130 may have one side touching the skin of a patient and forming a lesion based on vibration energy beneath the skin surface of the patient, thereby improving the skin. For example, a lesion may be formed as subcutaneous tissue is destroyed by vibration energy or the temperature of the tissue increases due to the vibration energy. The skin may be improved in a process in which the destroyed tissue of the patient heals. Also, since the destroyed tissue is fat tissue, and the fat tissue is destroyed by the hand piece 130, there may be an effect of reducing subcutaneous fat of the skin. Components included in the hand piece 130 will be described in detail below. The hand piece 130 may include a base part 133 formed on one side of the hand piece 130 and including a transducer arrangement surface 310. The base part 133 may be formed toward the skin of the patient. The components included in the hand piece 130 will be described in detail below.

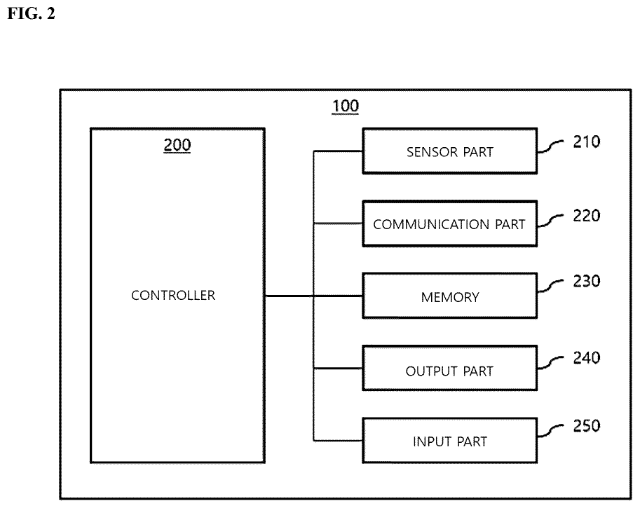

FIG. 2 is a view illustrating a block diagram of various components that may be included in the medical skin treatment apparatus according to one embodiment of the present disclosure.

The medical skin treatment apparatus 100 may include a sensor part 210, a communication part 220, a memory 230, an output part 240, an input part 250, and the controller 200.

The medical skin treatment apparatus 100 may include the controller 200. The controller 200 may control an operation of the medical skin treatment apparatus 100. For example, the medical skin treatment apparatus 100 may include the controller 200 for controlling an operation of at least one of the hand piece and a wheel allowing the main body 110 to navigate. The controller 200 may include a single processor or include a plurality of processors. The controller 200 may be included in the main body 110. When the controller 200 includes a plurality of processors, at least some of the plurality of processors may be provided at positioned physically spaced apart from the main body 110. Also, the medical skin treatment apparatus 100 is not limited thereto and may be implemented in various other manners.

According to one embodiment of the present disclosure, the controller 200 may control an operation of the medical skin treatment apparatus 100. For example, the medical skin treatment apparatus 100 may include a plurality of actuators, and the medical skin treatment apparatus 100 may control operations of the plurality of actuators to control an operation of the medical skin treatment apparatus 100. For example, the controller 200 may control an operation of the hand piece 130 to perform an operation for improving the skin of a patient.

The medical skin treatment apparatus 100 may include the sensor part 210. The sensor part 210 may obtain various pieces of information using at least one sensor. The sensor part 210 may be provided as a sensor using a measurement means such as a pressure measurement means, a potential measurement means, and an optical measurement means. For example, the sensor part 210 may include at least one of a distance measurement sensor or an encoder. Also, the sensor may include a pressure sensor, an infrared sensor, a light emitting diode (LED) sensor, a touch sensor, and the like. However, the present disclosure is not limited thereto. The sensor part may be included in at least one of the main body 110, the conveyor 120, and the hand piece.

Also, the medical skin treatment apparatus 100 may include the communication part 220. The communication part 220 may be a component allowing the medical skin treatment apparatus 100 to communicate with an internal module or an external device via a wire or wirelessly. The external device may be an external server or a user terminal. The user terminal may be a personal computer (PC), a smartphone, a tablet, or a wearable device. The communication part 220 may include a wired/wireless communication module for network connection. For example, wireless LAN (WLAN) (Wi-Fi), Wireless broadband (Wibro), World Interoperability for Microwave Access (Wimax), High Speed Downlink Packet Access (HSDPA), or the like may be used as wireless communication technology. For example, Digital Subscriber Line (XDSL), Fibers to the home (FTTH), Power Line Communication (PLC), or the like may be used as wired communication technology. Also, a network connector may include a short-range communication module to transmit and receive data to and from any device/terminal located at a short distance. For example, Bluetooth, Radio Frequency Identification (RFID), Infrared Data Association (IrDA), Ultra-Wideband (UWB), ZigBee, or the like may be used as short-range communication technology, but the present disclosure is not limited thereto.

The medical skin treatment apparatus 100 may include the memory 230. The controller 200 may execute instructions stored in the memory 230. The memory 230 may be included in the controller 200 or may be present outside the controller 200. The memory 230 may store various pieces of information related to the medical skin treatment apparatus 100. For example, the memory 230 may include information related to an operation method of the hand piece 130 and include captured images and user authentication information, but the present disclosure is not limited thereto.

The memory 230 may be implemented using nonvolatile storage media that can continuously store arbitrary data. Examples of the memory 230 may include a storage device based on a flash memory and/or a battery backup memory as well as a disk, an optical disc, and a magneto-optical storage device, but the present disclosure is not limited thereto. The memory 230 may be a volatile storage device which is a main storage device directly accessed by a processor and in which stored information is instantaneously erased once the power is turned off, such as a random access memory (RAM) such as a dynamic random access memory (DRAM) or a static random access memory (SRAM), but the memory 230 is not limited thereto. The memory 230 may be operated by the controller 200.

Also, the medical skin treatment apparatus 100 may further include a manipulation part providing an interface for manipulation of the medical skin treatment apparatus 100. The manipulation part may include the output part 240 and the input part 250.

The output part 240 may, under control of the controller 200, output treatment-related information, patient-related information, information related to control of the hand piece, and information related to a state of the main body as sound and images. The output part 240 may include a speaker or a display. The output part 240 may output a medical image generated by the controller 200. The output part 240 may output information necessary for a user to manipulate the medical skin treatment apparatus 100, such as a user interface (UI), user information, or subject information. Examples of the output part 240 may include a speaker, a printer, a cathode ray tube (CRT) display, a liquid crystal display (LCD), a plasma display panel (PDP), an organic light emitting diode (OLED) display, a field emission display (FED), an LED display, a vacuum fluorescent display (VFD), a digital light processing (DLP) display, a flat panel display (FPD), a 3D display, a transparent display, and the like and may include various other output devices within the scope self-evident to those of ordinary skill in the art.

The output part 240 may display treatment-related information. The treatment-related information may include at least one of a method of treatment to be provided to a patient, a treatment time, and information related to a disease of the patient. The method of treatment may include at least one of ultrasound treatment, radiofrequency (RF) treatment, and drug injection. The output part 240 may include patient-related information. The patient-related information may include at least one of the age and sex of the patient, a treatment period of the patient, and information related to a disease of the patient. The information related to control of the hand piece may include at least one of a button for controlling the hand piece, an operation cycle, the type of base part currently coupled to the hand piece, the intensity of energy used in treatment, and the frequency thereof. The information related to a state of the main body may include at least one of a battery state, a power connection state, and an error state of the main body.

Also, the output part 240 may output information related to control of the hand piece 130. For example, the controller 200 may control the output part 240 to output sound or light when the hand piece 130 is in operation to make one around the hand piece be aware that the hand piece is in operation.

The medical skin treatment apparatus 100 may be connected to a workstation via a wire or wirelessly. The workstation may be present in a space physically separated from the medical skin treatment apparatus 100.

The workstation may include a storage server. The storage server may store at least one of patient-related information, treatment-related information, and user (medical staff)-related information. The workstation may include a review device. The review device may receive a medical image from the storage server based on a command of the user and display the medical image. The workstation and the medical skin treatment apparatus 100 may send, store, process, and output data according to the Digital Imaging and Communications in Medicine (DICOM) standard. Also, the workstation may include a Picture Archiving and Communication System (PACS).

The workstation may include an output part, an input part, and a controller. The output part and the input part provide an interface for manipulation of the workstation and the medical skin treatment apparatus 100 to the user. The controller of the workstation may control the workstation and the medical skin treatment apparatus 100.

The medical skin treatment apparatus 100 may be controlled through the workstation or may be controlled by the controller 200 included in the medical skin treatment apparatus 100. Therefore, the user may control the medical skin treatment apparatus 100 through the workstation or may control the medical skin treatment apparatus 100 through the manipulation part and the controller 200 included in the medical skin treatment apparatus 100. In other words, the user may remotely control the medical skin treatment apparatus 100 through the workstation or may directly control the medical skin treatment apparatus 100.

The controller of the workstation and the controller 200 of the medical skin treatment apparatus 100 may be separate, but the present disclosure is not limited thereto. The controller of the workstation and the controller 200 of the medical skin treatment apparatus 100 may be implemented as a single integrated controller, and the integrated controller may be included in only one of the workstation and the medical skin treatment apparatus 100. Hereinafter, the controller 200 may be the controller of the workstation and/or the controller of the medical skin treatment apparatus 100.

The output part and the input part of the workstation and the output part 240 and the input part 250 of the medical skin treatment apparatus 100 may each provide an interface for manipulation of the medical skin treatment apparatus 100 to the user. The workstation and the medical skin treatment apparatus 100 may each include an output part and an input part, but the present disclosure is not limited thereto. The output part or input part may also be implemented in only one of the workstation and the medical skin treatment apparatus 100.

Hereinafter, the input part 250 is the input part of the workstation and/or the input part of the medical skin treatment apparatus 100, and the output part 240 is the output part of the workstation and/or the output part of the medical skin treatment apparatus 100.

The input part 250 may receive a command for manipulation of the medical skin treatment apparatus 100 and various pieces of information related to X-ray imaging from the user. The controller 200 may control or manipulate the medical skin treatment apparatus 100 based on the information input through the input part 250. Examples of the input part 250 may include a joystick, a keyboard, a mouse, a touchscreen, an imaging button, an unlocking button, a voice recognizer, a fingerprint recognizer, an iris recognizer, and the like and may include other input devices self-evident to those of ordinary skill in the art. The user may input a command for an operation of the hand piece through the input part 250, and a switch for such command input may be provided on the input part 250.

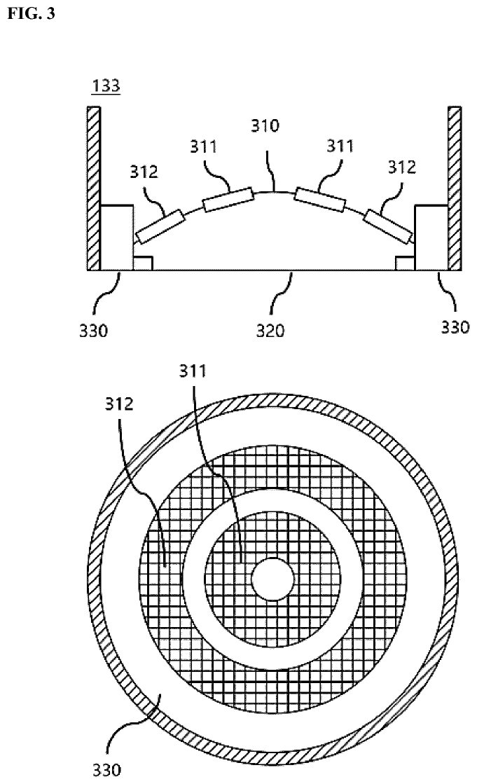

FIG. 3 is a view illustrating a cross-section of a base part according to one embodiment of the present disclosure.

Referring to FIG. 3, the base part 133 may include the transducer arrangement surface 310. The transducer arrangement surface 310 may have a surface that is flat relative to the skin of a patient.

The base part 133 may include a skin contact surface 320 which is a surface coming into contact with the skin of the patient. The skin contact surface 320 may be made of an elastic material and help the base part 133 and the skin come into close contact with each other.

The base part 133 may include a plurality of transducers 311 and 312 each having a ring shape. The plurality of transducers 311 and 312 may be formed on the transducer arrangement surface 310 that is concave. The plurality of transducers 311 and 312 may transmit vibration energy to the skin of the patient by vibrating at a predetermined treatment frequency. The predetermined treatment frequency may be 200 KHz or more and 20 MHz or less. The plurality of transducers 311 and 312 may include a first transducer 311 and a second transducer 312 having a greater diameter than the first transducer 311.

The plurality of transducers 311 and 312 may have different diameters. The plurality of transducers 311 and 312 each having a ring shape may form concentric circles. The plurality of transducers 311 and 312 may simultaneously treat different layers beneath the skin by having a plurality of focal points each formed vertically beneath the skin from a center of the concentric circles. Depths of the focal points of the plurality of transducers 311 and 312 that are formed beneath the skin may be proportional to the diameters of the plurality of transducers 311 and 312. The greater the diameters of the plurality of transducers 311 and 312, the deeper the depths of the focal points may be. The second transducer 312 may have a greater diameter than the first transducer 311. Therefore, the depth of the focal point of the second transducer 312 may be deeper than the depth of the focal point of the first transducer 311. Here, the depth of the focal point may be a distance from a center of the skin contact surface 320 to a point to which vibration energy is concentrated. The medical skin treatment apparatus 100 may be able to perform more effective treatment by simultaneously treating different layers beneath the skin of a patient. On the transducer arrangement surface 310, the plurality of transducers 311 and 312 may be formed so that the second transducer 312 having a greater diameter comes into contact with the first transducer 311 by surrounding the first transducer 311 along a circumference of the first transducer 311. Also, the plurality of transducers 311 and 312 may be formed to be spaced from and not come into contact with each other.

The base part 133 may include a thermoelectric device 330. The thermoelectric device 330 may be formed on the transducer arrangement surface 310 that is concave. The thermoelectric device 330 may be formed along outer circumferential surfaces of the plurality of transducers 311 and 312. The thermoelectric device 330 may be formed along an edge of the transducer arrangement surface 310. The thermoelectric device 330 may come into contact with at least one of the transducer arrangement surface 310, the skin contact surface 320, and the plurality of transducers 311 and 312. The thermoelectric device 330 may cool at least one of the transducer arrangement surface 310, the skin contact surface 320, and the plurality of transducers 311 and 312 in contact therewith. A cooling surface of the thermoelectric device 330 may be in contact with at least one of the plurality of transducers 311 and 312. The thermoelectric device 330 may cool at least one of the plurality of transducers 311 and 312 in contact therewith to prevent the skin surface of a patient, which touches one side of the hand piece 130, from getting burned. Also, the thermoelectric device may be directly formed on the base part 133 along an inner wall of the base part 133. The cooling surface of the thermoelectric device 330 may face the skin. The cooling surface of the thermoelectric device 330 may be in contact with the skin together with the skin contact surface 320. The cooling surface of the thermoelectric device 330 that is in contact with the skin may be cooled to prevent the skin surface of the patient from getting burned.

The plurality of transducers 311 and 312 may simultaneously or sequentially radiate vibration energy based on an input of a user relating to a treatment area. The medical skin treatment apparatus 100 may store skin information relating to the treatment area input by the user. For example, the hand piece 130 may include a sensor measuring a skin thickness. The medical skin treatment apparatus 100 may determine a skin thickness of a treatment target site based on at least one of the skin thickness input by the user and the skin thickness measured by the sensor. When the skin thickness of the treatment target site is a predetermined thickness or more, the first transducer 311 and the second transducer 312 may simultaneously radiate vibration energy. When the skin thickness is less than the predetermined thickness, only one of the first transducer 311 and the second transducer 312 may radiate vibration energy. The medical skin treatment apparatus 100 may control operations of the plurality of transducers 311 and 312 according to the skin thickness to prevent treatment from being performed near nervous tissue and reduce pain of the patient during the treatment.

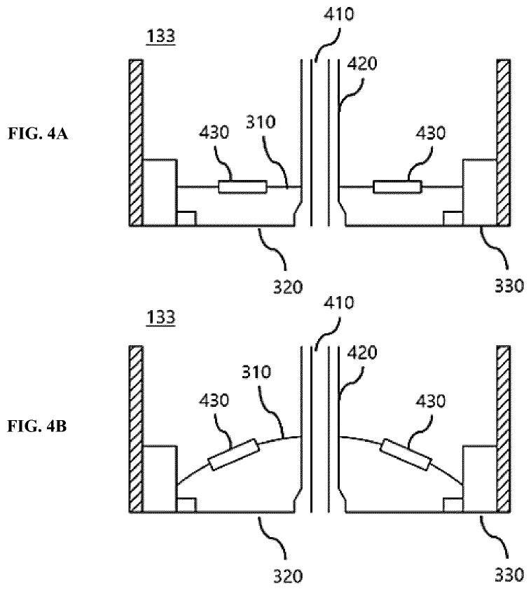

FIGS. 4A and 4B are views illustrating a cross-section of the base part according to one embodiment of the present disclosure.

The hand piece 130 may include a drug injection part 410 injecting a drug into the skin of a patient. The drug injection part 410 may be coupled to the drug supply through a rubber tube. The drug injection part 410 may be located at a center of the base part 133. The drug injection part 410 may pass through the transducer arrangement surface 310 and the skin contact surface 320. One side of the drug injection part 410 may come into contact with the skin of the patient. The drug injection part 410 may spray a drug received from the drug supply to a point in contact with the skin of the patient.

The hand piece 130 may include a skin suction part 420 suctioning the skin of the patient. The skin suction part 420 may be formed along an outer circumferential surface of the drug injection part 410. The skin suction part 420 and the drug injection part 410 may form concentric circles. The skin suction part 420 may have a form surrounding a circumference of the drug injection part 410. For example, a tube forming the drug injection part 410 may be formed inside a tube forming the skin suction part 420.

Referring to FIG. 4A, the transducer arrangement surface 310 may have a surface that is flat relative to the skin of a patient. The transducer arrangement surface 310 may include a transducer 430 having a ring shape and transmitting vibration energy to the skin of the patient by vibrating at a predetermined treatment frequency. The transducer 430 may be formed along an outer circumferential surface of the skin suction part 420. The transducer 430 included in the transducer arrangement surface 310 having the flat surface may help the drug of the drug injection part 410 to be rapidly absorbed into the skin. Also, the transducer 430 included in the transducer arrangement surface 310 having the flat surface may treat the skin by emitting vibration energy. For example, due to constructive interference of vibration emitted by the transducer, high vibration energy may be formed at a specific site beneath the skin, and a lesion may be formed.

Referring to FIG. 4B, the transducer arrangement surface 310 may have a surface that is concave relative to the skin of the patient. Due to the transducer arrangement surface 310 having the concave surface, the transducer 430 may emit vibration energy in a direction perpendicular to a direction of inclination of the concave surface. Therefore, the transducer 430 may form a focal point intensively on a central axis of the transducer arrangement surface 310.

At least one of the drug injection part 410 and the skin suction part 420 may form a concentric circle with at least one of the transducer 430 and the thermoelectric device 330. By at least one of the drug injection part 410 and the skin suction part 420 forming a concentric circle with the transducer 430, an area in which the transducer 430 forms a focal point and an area in which the drug injection part 410 injects a drug may be located on the same axis, and the drug may be accurately injected into a treatment site.

The skin suction part 420 may suction the skin of the patient and form a negative pressure. At least one of the main body 110 and the hand piece 130 may include a compressor. When the compressor is formed on the main body 110, the compressor may be coupled to the skin suction part 420 through a tube included in the cable 131. During treatment, the skin suction part 420 may suction the skin and fix the hand piece 130 and the treatment target site in a state in which they are in close contact with each other. Therefore, an influence of movement that may affect the treatment, such as shaking of a hand of the user performing the treatment using the hand piece 130 and movement of the patient, may be minimized, and more precise treatment may be possible.

The main body 110 or the hand piece 130 may include a pressure sensor for measuring a pressure formed inside the skin suction part 420. Based on the pressure sensor, the controller 200 may measure the pressure formed inside the skin suction part 420. When the pressure formed inside the skin suction part 420 is a critical pressure or more, the controller 200 may control drug injection by the drug injection part 410 to stop. Also, when the pressure formed inside the skin suction part 420 is the critical pressure or more, the controller 200 may control the transducer 430 to not emit vibration energy. The critical pressure may be a pressure value sufficient for suctioning the skin and fixing the treatment site. The critical pressure may be a predetermined value. When the pressure formed inside the skin suction part 420 rises to the critical pressure or more during treatment, the controller 200 may control at least one of stoppage of drug injection and stoppage of vibration energy emission to be performed. The controller 200 may control warning sound and a warning message, which notify that the hand piece 130 needs to be brought into close contact with the skin again, to be output through the output part 240.

When the pressure of the skin suction part 420 is less than the critical pressure, the controller 200 may control the drug injection part 410 to inject a drug. When the pressure of the skin suction part 420 is less than the critical pressure, the controller 200 may control the transducer 430 to emit vibration energy. By the controller 200 controlling vibration energy to be emitted only when the hand piece 130 is in close contact with the skin, treatment may be safely performed.

However, the present disclosure is not limited thereto, and the hand piece 130 may include a proximity sensor. The proximity sensor may be a sensor measuring a distance between the hand piece and the skin of a patient or determining whether the hand piece and the skin of the patient are close enough to provide treatment. When the proximity sensor indicates that the distance between the skin of the patient and the hand piece 130 is a predetermined critical distance or more, the controller 200 may control drug injection by the drug injection part 410 to stop. Also, when the proximity sensor indicates that the distance between the skin of the patient and the hand piece 130 is less than the predetermined critical distance, the controller 200 may control drug injection by the drug injection part 410 to be performed.

The hand piece 130 may include a filter for filtering a drug and waste matter suctioned by the skin suction part 420. The filter may be located between the compressor and the skin suction part 420. The filter may be replaced by a user. The filter may allow passage of air between the skin suction part 420 and the compressor and block passage of the drug and the waste matter. The filter may prevent the drug and the waste matter suctioned by the skin suction part 420 from entering the compressor and the hand piece. Management of the hand piece 130 may be facilitated by the filter included in the hand piece 130. The user may maintain the hand piece 130 clean just by replacing the filter.

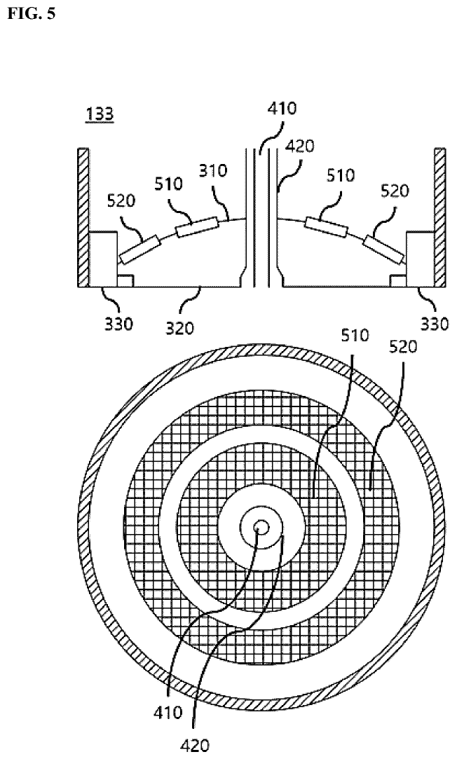

FIG. 5 is a view illustrating a cross-section of the base part according to one embodiment of the present disclosure.

Referring to FIG. 5, a plurality of transducers 510 and 520, the skin suction part 420, the drug injection part 410, and the thermoelectric device 330 may be simultaneously formed in the hand piece 130. The base part 133 may include the transducer arrangement surface 310 having a surface that is concave relative to the skin of a patient. The transducer arrangement surface 310 may be located above the skin contact surface 320. The plurality of transducers 510 and 520 of FIG. 5 may include the same configuration as the plurality of transducers 311 and 312 of FIG. 3. The drug injection part 410 and the skin suction part 420 of FIG. 5 may include the same configuration as the drug injection part 410 and the skin suction part 420 of FIG. 4.

The plurality of transducers 510 and 520, the skin suction part 420, the drug injection part 410, and the thermoelectric device 330 may form concentric circles. The plurality of transducers 510 and 520 may, by forming concentric circles with each other, form a plurality of focal points on upper and lower portions of the central axis of the transducer arrangement surface 310. The plurality of focal points of the plurality of transducers 510 and 520 may intensively and simultaneously treat different layers beneath the skin. By the drug injection part 410 and the skin suction part 420 forming concentric circles with the plurality of transducers 510 and 520, an area in which the plurality of transducers 510 and 520 form focal points and an area in which the drug injection part 410 injects a drug may coincide with each other, and the drug may be accurately injected into a treatment site.

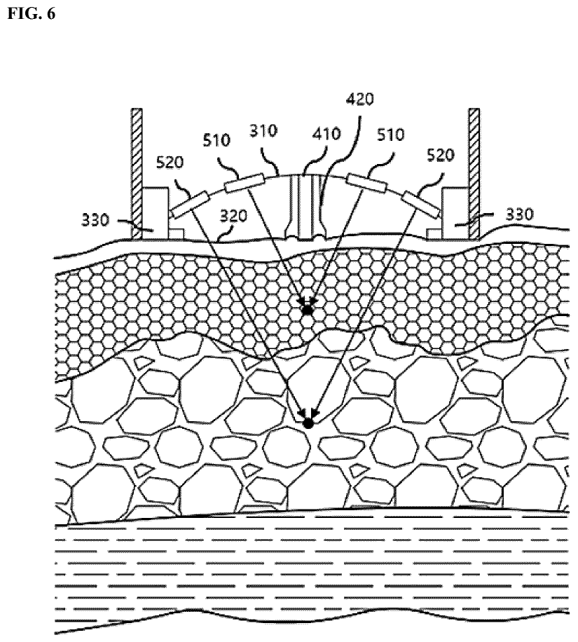

FIG. 6 is a view for describing a hand piece according to one embodiment of the present disclosure.

A process in which the medical skin treatment apparatus provides ultrasound treatment according to various embodiments of the present disclosure will be described below with reference to FIGS. 5 and 6.

FIG. 7 is a flowchart for describing an operation of the medical skin treatment apparatus according to one embodiment of the present disclosure.

The controller 200 may perform obtaining a cumulative number of shots of the plurality of transducers 510 and 520 (710). The cumulative number of shots may be a cumulative value of the number of shots provided by at least one of a first transducer 510 and a second transducer 520 to a patient after the start of treatment. The cumulative number of shots may be obtained for each of the first transducer 510 and the second transducer 520. The controller 200 may control at least one of the first transducer 510 and the second transducer 520 based on the cumulative number of shots of the first transducer 510. The controller 200 may control at least one of the first transducer 510 and the second transducer 520 based on the cumulative number of shots of the second transducer 520. The controller 200 may measure time from the start of the treatment. The start of the treatment may be when at least one of the plurality of transducers 510 and 520 starts to emit vibration energy. The controller 200 may also obtain time at which a corresponding shot is provided for each shot provided after the start of the treatment.

The controller 200 may perform obtaining a skin temperature relative to the cumulative number of shots based on at least one of a first table and a first function, in which the cumulative number of shots and the skin temperature are matched (720). The controller 200 may obtain the skin temperature by applying the cumulative number of shots to at least one of the first table and the first function. The first table may include a first-first table and a first-second table. The first function may include a first-first function and a first-second function. The controller 200 may store at least one of the first-first table and the first-first function, in which the cumulative number of shots and the skin temperature of a patient are matched. At least one of the first-first table and the first-first function may be based on the following function. For example, the first-first table may be a table in which values of T1 relative to s and TO are matched based on the following equation.

$$A 1 * s + T 0 = T 1$$

However, the present disclosure is not limited thereto, and at least one of the first-first table and the first-first function may be based on the following function.

$$A 1 * log ( s ) + T 0 = T 1$$

Here, s may be the cumulative number of shots. T0 may be an initial skin temperature. Upon the start of treatment, T0 may be determined as at least one of a predetermined temperature and the room temperature. T1 may be the current skin temperature. A previous T1 may be used as T0 for determining the current skin temperature of a subsequent point in time. A1 may be a predetermined positive real number that indicates a heating slope of the skin caused by a transducer. A1 may be obtained using the following equation.

$$A 1 = k / V + b$$

Here, V may be a voltage applied to the thermoelectric device 330. k may be a predetermined positive real number. b may be a predetermined constant. A1 may be inversely proportional to the voltage applied to the thermoelectric device 330.

However, the present disclosure is not limited thereto, and A1 may be obtained using the following equation.

$$A 1 = k * V + b$$

Here, k may be a predetermined negative real number. A value of A1 may decrease with an increase in the voltage applied to the thermoelectric device 330.

The skin temperature of the patient may increase with an increase in the cumulative number of shots. The controller 200 may obtain the skin temperature by matching the cumulative number of shots to at least one of the first-first table and the first-first function. The controller 200 may store at least one of the first-second table and the first-second function, in which the skin temperature of the patient according to natural cooling is matched. When treatment is paused in the middle, the controller 200 may obtain a time at which the treatment is paused and a time at which the treatment is resumed. The controller 200 may obtain a time during which the treatment is paused. The time during which the treatment is paused may be the time from the time at which the treatment is paused until the time at which the treatment is resumed. At least one of the first-second table and the first-second function may be based on the following function.

$$B 1 * t + T 0 = T 1$$

However, the present disclosure is not limited thereto, and at least one of the first-second table and the first-second function may be based on the following function.

$$B 1 / t + T 0 = T 1$$

Here, B1 may be a predetermined cooling slope according to natural cooling. B1 may be a predetermined negative real number. T0 may be a skin temperature at the time at which the treatment is paused. t may be the time during which the treatment is paused. T1 may be a skin temperature at the time at which the treatment is resumed.

The controller 200 may obtain the skin temperature of the patient by matching the time during which the treatment is paused to at least one of the first-second table and the first-second function. The skin temperature may decrease with an increase in the time during which the treatment is paused. The treatment may be resumed, and the controller 200 may use the skin temperature obtained using at least one of the first-second table and the first-second function as an initial skin temperature of at least one of the first-first table and the first-first function.

Step (720) may be automatically performed every predetermined time during the treatment to automatically obtain the skin temperature of the patient in contact with the hand piece 130. The medical skin treatment apparatus 100 may display the obtained skin temperature in real time on the display of the output part 240.

The controller 200 may perform determining the number of treatment shots based on the skin temperature relative to the cumulative number of shots (730). The number of treatment shots may be the number of shots to be provided to the patient from now on by at least one of the first transducer 510 and the second transducer 520. The number of treatment shots may be the number of shots per predetermined time of at least one of the first transducer 510 and the second transducer 520. The number of treatment shots may be determined for each of the first transducer 510 and the second transducer 520. The controller 200 may store data of the number of treatment shots according to the skin temperature. The controller 200 may adjust the number of treatment shots according to the skin temperature obtained in Step (720). The number of treatment shots may decrease with an increase in the skin temperature and may increase with a decrease in the skin temperature. However, the present disclosure is not limited thereto, and the number of treatment shots may be constant regardless of an increase or decrease of the skin temperature.

FIG. 8 is a flowchart for describing an operation of the medical skin treatment apparatus according to one embodiment of the present disclosure.

The controller 200 may perform obtaining a cumulative radiation time of the plurality of transducers 510 and 520 (810). The cumulative radiation time may be a cumulative value of the time during which at least one of the first transducer 510 and the second transducer 520 radiates ultrasonic waves provided to the patient after the start of the treatment. The cumulative radiation time may be obtained for each of the first transducer 510 and the second transducer 520. The controller 200 may control at least one of the first transducer 510 and the second transducer 520 based on the cumulative radiation time of the first transducer 510. The controller 200 may control at least one of the first transducer 510 and the second transducer 520 based on the cumulative radiation time of the second transducer 520. The controller 200 may measure time from the start of the treatment. The controller 200 may obtain a time at which radiation of ultrasonic waves starts and a time at which the radiation ends.

The controller 200 may perform obtaining the skin temperature relative to the cumulative radiation time based on at least one of the second table and the second function, in which the cumulative radiation time and the skin temperature are matched (820). The controller 200 may obtain the skin temperature by applying the cumulative radiation time to at least one of the second table and the second function. The second table may include a second-first table and a second-second table. The second function may include a second-first function and a second-second function. The controller 200 may store at least one of the second-first table and the second-first function, in which the cumulative radiation time and the skin temperature are matched. At least one of the second-first table and the second-first function may be based on the following function.

$$A 2 * r + T 0 = T 1$$

However, the present disclosure is not limited thereto, and at least one of the second-first table and the second-first function may be based on the following function.

$$A 2 * log ( r ) + T 0 = T 1$$

Here, r may be a cumulative radiation time. T0 may be an initial skin temperature. T1 may be the current skin temperature. A2 may be a predetermined positive real number that indicates a heating slope of the skin caused by a transducer. A2 may be obtained using the following equation.

$$A 2 = k / V + b$$

Here, V may be a voltage applied to the thermoelectric device 330. k may be a predetermined positive real number. b may be a predetermined constant. A2 may be inversely proportional to the voltage applied to the thermoelectric device 330.

However, the present disclosure is not limited thereto, and A2 may be obtained using the following equation.

$$A 2 = k * V + b$$

Here, k may be a predetermined negative real number. A value of A2 may decrease with an increase in the voltage applied to the thermoelectric device 330.

The skin temperature of the patient may increase with an increase in the cumulative radiation time. The controller 200 may obtain the skin temperature by matching the cumulative radiation time to at least one of the second-first table and the second-first function.

The controller 200 may store at least one of the second-second table and the second-second function, in which the skin temperature of the patient according to natural cooling is matched. When treatment is paused in the middle, the controller 200 may obtain a time at which the treatment is paused and a time at which the treatment is resumed. The controller 200 may obtain a time during which the treatment is paused. The time during which the treatment is paused may be the time from the time at which the treatment is paused until the time at which the treatment is resumed. At least one of the second-second table and the second-second function may be based on the following function.

$$B 2 * t + T 0 = T 1$$

However, the present disclosure is not limited thereto, and at least one of the second-second table and the second-second function may be based on the following function.

$$B 2 / t + T 0 = T 1$$

Here, B2 may be a predetermined cooling slope according to natural cooling. B2 may be a predetermined negative real number. T0 may be a skin temperature at the time at which the treatment is paused. t may be the time during which the treatment is paused. T1 may be a skin temperature at the time at which the treatment is resumed.

The controller 200 may obtain the skin temperature of the patient by matching the time during which the treatment is paused to at least one of the second-second table and the second-second function. The skin temperature may decrease with an increase in the time during which the treatment is paused. The treatment may be resumed, and the controller 200 may use the skin temperature obtained using at least one of the second-second table and the second-second function as an initial skin temperature of at least one of the second-first table and the second-first function.

Step (820) may be automatically performed every predetermined time during the treatment to automatically obtain the skin temperature of the patient in contact with the hand piece 130. The medical skin treatment apparatus 100 may display the obtained skin temperature in real time on the display of the output part 240.

The controller 200 may perform determining a treatment radiation time based on the skin temperature relative to the cumulative radiation time (830). The treatment radiation time may be a radiation time of ultrasonic waves to be provided to the patient from now on by the first transducer 510 and the second transducer 520. The treatment radiation time may be determined for each of the first transducer 510 and the second transducer 520. The controller 200 may store data of the treatment radiation time according to the skin temperature. The controller 200 may adjust the treatment radiation time according to the skin temperature obtained in Step (820). The treatment radiation time may decrease with an increase in the skin temperature and may increase with a decrease in the skin temperature. However, the present disclosure is not limited thereto, and the treatment radiation time may be constant regardless of an increase or decrease of the skin temperature.

Steps (710, 720, 730) of FIG. 7 and Steps (810, 820, 830) of FIG. 8 may be performed individually or simultaneously. When Steps (710, 720, 730) of FIG. 7 and Steps (810, 820, 830) of FIG. 8 are performed simultaneously, the controller 200 may obtain the skin temperature obtained in Step (720) of FIG. 7 and the skin temperature obtained in Step (820) of FIG. 8. The controller 200 may obtain a representative skin temperature using the skin temperature of Step (720) and the skin temperature of Step (820). The representative skin temperature may be one of a mean value, a median value, a maximum value, and a minimum value of the skin temperature of Step (720) and the skin temperature of Step (820). The controller 200 may display the representative skin temperature on the output part 240. The controller 200 may obtain the number of treatment shots and the treatment radiation time by performing Step (730) and Step (830) using the representative skin temperature.

The controller 200 may include information on the time sufficient for the plurality of transducers 510 and 520 to be naturally cooled. When the corresponding time passes, the controller 200 may reset the cumulative number of shots and the cumulative radiation time.

The controller 200 may automatically adjust the temperature of the thermoelectric device 330 by controlling the voltage applied to the thermoelectric device 330. Due to the Peltier effect, the temperature of the cooling surface of the thermoelectric device 330 may decrease with an increase in the voltage applied to the thermoelectric device 330. The temperature of the cooling surface of the thermoelectric device 330 may increase with a decrease in the voltage applied to the thermoelectric device 330.

The controller 200 may determine the voltage to be applied to the thermoelectric device 330 based on at least one of the skin temperature obtained from at least one of the first table, the first function, the second table, and the second function, and a third table and a third function in which the skin temperature and the voltage applied to the thermoelectric device 330 are matched. However, the present disclosure is not limited thereto, and the controller 200 may also determine the voltage to be applied to the thermoelectric device 330 based on at least one of the skin temperature obtained by direct measurement, the third table, and the third function.

The third table may include a third-first table and a third-second table. The third function may include a third-first function and a third-second function. The controller 200 may store at least one of the third-first table and the third-first function, in which the skin temperature and the voltage applied to the thermoelectric device 330 are matched. At least one of the third-first table and the third-first function may be based on the following function.

$$C 1 * T + V 0 = V 1$$

However, the present disclosure is not limited thereto, and at least one of the third-first table and the third-first function may be based on the following function.

$$C 1 * log ( T ) + V 0 = V 1$$

Here, T may be a skin temperature. V0 may be an initial voltage. V1 may be a voltage to be applied to the thermoelectric device 330. C1 may be a predetermined positive real number that indicates a heating slope of the skin temperature.

Also, at least one of the third-first table and the third-first function may be based on the following function.

$$C 1 / T + Vlimit = V 1$$

Here, C1 may be a predetermined negative real number that indicates a heating slope of the skin temperature. T may be a skin temperature. Vlimit may be a predetermined constant. V1 may be a voltage to be applied to the thermoelectric device 330.

The voltage applied to the thermoelectric device 330 may increase with an increase in the skin temperature. The first table, the first function, the second table, and the second function may include skin temperature data that takes cooling of the thermoelectric device 330 into consideration. Therefore, the controller 200 may determine the voltage to be applied to the thermoelectric device 330 by applying the skin temperature obtained from at least one of the first table, the first function, the second table, and the second function to at least one of the third-first table and the third-first function. Since the voltage to be applied to the thermoelectric device 330 increases with an increase in the skin temperature, the skin temperature may not increase to a predetermined temperature or more. Therefore, the skin of the patient may not get burned.

The controller 200 may store at least one of the third-second table and the third-second function, in which the voltage applied to the thermoelectric device 330 according to natural cooling is matched. When treatment is paused in the middle, the controller 200 may obtain a time at which the treatment is paused and a time at which the treatment is resumed. The controller 200 may obtain a time during which the treatment is paused. The time during which the treatment is paused may be the time from the time at which the treatment is paused until the time at which the treatment is resumed. At least one of the third-second table and the third-second function may be based on the following function.

$$C 2 * t + V 1 = V 2$$

Here, C2 may be a predetermined negative real number that indicates a cooling slope according to natural cooling. V1 may be an initial voltage of the thermoelectric device 330. V2 may be a voltage to be applied to the thermoelectric device 330. t may be the time during which the treatment is paused.

However, the present disclosure is not limited thereto, and at least one of the third-second table and the third-second function may be based on the following function.

$$C 2 / ( t + a ) + V 1 - C 2 / a = V 2$$

Here, C2 may be a predetermined positive real number that indicates a cooling slope according to natural cooling. t may be the time during which the treatment is paused. a may be a predetermined constant and may be a predetermined positive real number. V1 may be an initial voltage of the thermoelectric device 330. V2 may be a voltage to be applied to the thermoelectric device 330.

V2, which is the voltage to be applied to the thermoelectric device 330, may decrease with an increase in t, which is the time during which the treatment is paused.

As in the embodiments of FIGS. 7 and 8, the medical skin treatment apparatus 100 may obtain the skin temperature without having a temperature sensor. In this way, the size of the hand piece 130 may be reduced in the medical skin treatment apparatus 100. Also, since a temperature sensor is not used, the cost of the medical skin treatment apparatus 100 may be reduced. By automatically controlling the plurality of transducers 510 and 520 and the thermoelectric device 330 without using a temperature sensor, the medical skin treatment apparatus 100 may have the same skin improving effect and stability as an existing apparatus having a temperature sensor.

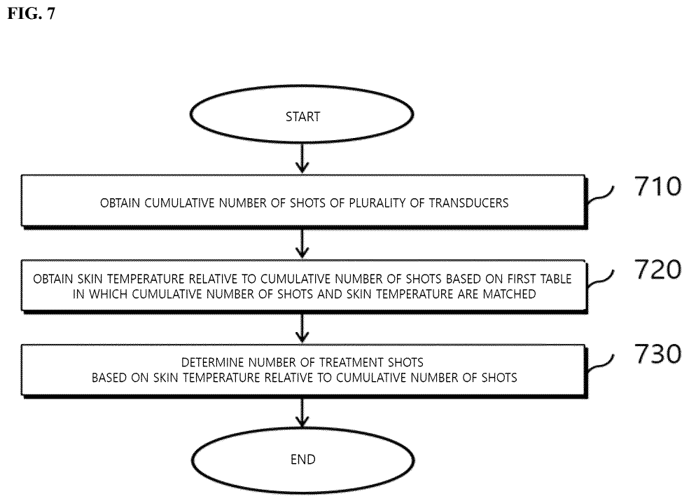

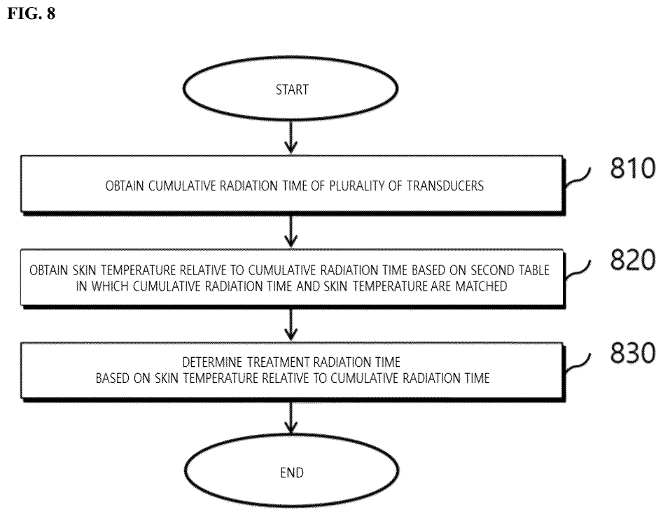

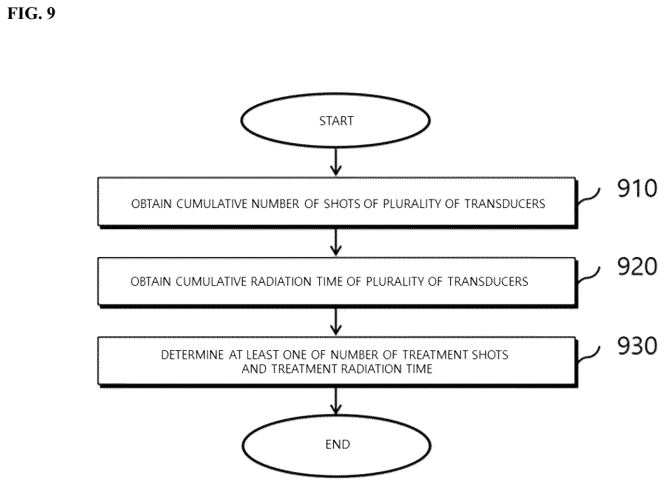

FIG. 9 is a flowchart for describing an operation of the medical skin treatment apparatus according to one embodiment of the present disclosure.

The controller 200 may perform obtaining the cumulative number of shots of the plurality of transducers 510 and 520 (910). The controller 200 may perform obtaining the cumulative radiation time of the plurality of transducers 510 and 520 (920). The controller 200 may perform determining at least one of the number of treatment shots and the treatment radiation time based on at least one of the cumulative number of shots and the cumulative radiation time (930). The controller 200 may store at least one of a fourth table and a fourth function, in which at least one of the cumulative number of shots and the cumulative radiation time is matched with at least one of the number of treatment shots and the treatment radiation time. Step (930) may be performed based on at least one of the fourth table and the fourth function. The fourth table may include a fourth-first table, a fourth-second table, a fourth-third table, and a fourth-fourth table. The fourth function may include a fourth-first function, a fourth-second function, a fourth-third function, and a fourth-fourth function.

The controller 200 may store at least one of the fourth-first table and the fourth-first function, in which the cumulative number of shots is matched with the number of treatment shots. At least one of the fourth-first table and the fourth-first function may be based on the following function.

$$A 3 / s = s 1$$

Here, s may be the cumulative number of shots. s1 may be the number of treatment shots. A3 may be a predetermined positive real number that indicates a heating slope.

However, the present disclosure is not limited thereto, and at least one of the fourth-first table and the fourth-first function may be based on the following function.

$$A 3 * s + s 0 = s 1$$

Here, s may be the cumulative number of shots. s1 may be the number of treatment shots. A3 may be a predetermined negative real number that indicates a heating slope. s0 may be an initial number of treatment shots. s1, which is the number of treatment shots, may decrease with an increase in s, which is the cumulative number of shots.

The controller 200 may store at least one of the fourth-second table and the fourth-second function, in which the cumulative radiation time is matched with the treatment radiation time. At least one of the fourth-second table and the fourth-second function may be based on the following function.

$$A 3 / r = r 1$$

Here, r may be the cumulative radiation time. r1 may be the treatment radiation time. A3 may be a predetermined positive real number that indicates a heating slope.

However, the present disclosure is not limited thereto, and at least one of the fourth-second table and the fourth-second function may be based on the following function.

$$A 3 * r + r 0 = r 1$$

Here, r may be the cumulative radiation time. r1 may be the treatment radiation time. A3 may be a predetermined negative real number that indicates a heating slope. r0 may be an initial treatment radiation time. r1, which is the treatment radiation time, may decrease with an increase in r, which is the cumulative radiation time.

The controller 200 may obtain at least one of the number of treatment shots and the treatment radiation time by matching at least one of the cumulative number of shots and the cumulative radiation time to at least one of the fourth-first table, the fourth-first function, the fourth-second table, and the fourth-second function. Therefore, the controller 200 may automatically adjust the number of treatment shots and the treatment radiation time without obtaining the skin temperature.

When treatment is paused for a moment, as time passes, natural cooling may occur, and the temperatures of the plurality of transducers 510 and 520 and the skin temperature may decrease. The controller 200 may obtain the time during which the treatment is paused. The controller 200 may store at least one of the fourth-third table and the fourth-third function. The fourth-third table and the fourth-third function may include the number of treatment shots according to the time during which the treatment is paused. The controller 200 may store at least one of the fourth-fourth table and the fourth-fourth function. The fourth-fourth table and the fourth-fourth function may include the treatment radiation time according to the time during which the treatment is paused. During the treatment resumed afterwards, the controller 200 may provide treatment using at least one of the number of treatment shots and the treatment radiation time that match at least one of the number of treatment shots and the treatment radiation time obtained using at least one of the fourth-first table, the fourth-first function, the fourth-second table, and the fourth-second function.

At least one of the fourth-third table and the fourth-third function may be based on the following function.

$$B 3 * t + s 0 = s 1$$

However, the present disclosure is not limited thereto, and at least one of the fourth-third table and the fourth-third function may be based on the following function.

$$B 3 * log ( t ) + s 0 = s 1$$

Here, B3 may be a predetermined positive real number that indicates a cooling slope according to natural cooling. t may be the time during which the treatment is paused. s0 may be an initial number of treatment shots. s1 may be the current number of treatment shots.

At least one of the fourth-fourth table and the fourth-fourth function may be based on the following function.

$$B 3 * t + r 0 = r 1$$

However, the present disclosure is not limited thereto, and at least one of the fourth-fourth table and the fourth-fourth function may be based on the following function.

$$B 3 * log ( t ) + r 0 = r 1$$

Here, B3 may be a predetermined positive real number that indicates a cooling slope according to natural cooling. t may be the time during which the treatment is paused. r0 may be an initial treatment radiation time. r1 may be the current treatment radiation time.

At least one of s1, which is the number of treatment shots, and r1, which is the treatment radiation time, may increase with an increase in the time during which the treatment is paused.

In the embodiment of FIG. 9, the medical skin treatment apparatus 100 may obtain at least one of the number of treatment shots and the treatment radiation time without obtaining the skin temperature. Since the medical skin treatment apparatus 100 does not obtain the skin temperature, a processing speed for providing at least one of the number of treatment shots and the treatment radiation time may increase.

According to various embodiments of the present disclosure, control of the thermoelectric device 330 may be performed along with the embodiments of FIGS. 7 to 9.

However, the present disclosure is not limited thereto, and control of the thermoelectric device 330 may be performed without carrying out the embodiments of FIGS. 7 to 9, and the medical skin treatment apparatus may maintain the temperature of the skin contact surface 320 of the hand piece within a predetermined critical temperature to prevent the skin surface of the patient from getting burned. A method of controlling the thermoelectric device 330 will be described below.

The controller 200 may automatically adjust the temperature of the thermoelectric device 330 by controlling the voltage applied to the thermoelectric device 330. The voltage applied to the thermoelectric device 330 may increase with an increase in the cumulative number of shots and the cumulative radiation time. For example, the controller 200 may determine the voltage to be applied to the thermoelectric device 330 based on the cumulative number of shots and the cumulative radiation time. At least one of the third table and the third function may include data of the voltage to be applied to the thermoelectric device 330 that matches at least one of the cumulative number of shots and the cumulative radiation time. The controller 200 may determine the voltage to be applied to the thermoelectric device 330 by applying at least one of the cumulative number of shots obtained in Step (910) of FIG. 9 and the cumulative radiation time obtained in Step (920) of FIG. 9 to at least one of the third table and the third function.

The third table may include a third-third table, a third-fourth table, and a third-fifth table. The third function may include a third-third function, a third-fourth function, and a third-fifth function.

The controller 200 may determine the voltage to be applied to the thermoelectric device 330 based on at least one of the third-third table and the third-third function, in which the cumulative number of shots and the voltage applied to the thermoelectric device 330 are matched. At least one of the third-third table and the third-third function may be based on the following function.

$$C 3 * s + V 0 = V 1$$

However, the present disclosure is not limited thereto, and at least one of the third-third table and the third-third function may be based on the following function.

$$C 3 * log ( s ) + V 0 = V 1$$

Here, C3 may be a predetermined positive real number that indicates a heating slope of the skin caused by a transducer. s may be the cumulative number of shots. V0 may be an initial voltage of the thermoelectric device 330. V1 may be the voltage to be applied to the thermoelectric device 330.

Also, at least one of the third-third table and the third-third function may be based on the following function.

$$C 3 / s + Vlimit = V 1$$

Here, C3 may be a predetermined negative real number that indicates a heating slope of the skin caused by a transducer. s may be the cumulative number of shots. Vlimit may be a predetermined constant. V1 may be the voltage to be applied to the thermoelectric device 330.

V1, which is the voltage to be applied to the thermoelectric device 330, may increase with an increase in s, which is the cumulative number of shots.

The controller 200 may determine the voltage to be applied to the thermoelectric device 330 based on at least one of the third-fourth table and the third-fourth function, in which the cumulative radiation time and the voltage applied to the thermoelectric device 330 are matched. At least one of the third-fourth table and the third-fourth function may be based on the following function.

$$C 3 * r + V 0 = V 1$$

However, the present disclosure is not limited thereto, and at least one of the third-fourth table and the third-fourth function may be based on the following function.

$$C 3 * log ( r ) + V 0 = V 1$$

Here, C3 may be a predetermined positive real number that indicates a heating slope of the skin caused by a transducer. r may be the cumulative radiation time. V0 may be an initial voltage of the thermoelectric device 330. V1 may be the voltage to be applied to the thermoelectric device 330. The initial voltage of the thermoelectric device may have a predetermined value when the medical skin treatment apparatus 100 is run for the very first time, and then V1 may become an initial voltage and be used in determining a subsequent V1.

Also, at least one of the third-fourth table and the third-fourth function may be based on the following function.

$$C 3 / r + Vlimit = V 1$$

Here, C3 may be a predetermined negative real number that indicates a heating slope of the skin caused by a transducer. r may be the cumulative radiation time. Vlimit may be a predetermined constant. V1 may be the voltage to be applied to the thermoelectric device 330. V1 may be obtained every predetermined time based on the above function or table, and the medical skin treatment apparatus 100 may control the thermoelectric device based on V1.

V1, which is the voltage to be applied to the thermoelectric device 330, may increase with an increase in r, which is the cumulative radiation time.

The controller 200 may store at least one of the third-fifth table and the third-fifth function, in which the skin temperature of the patient according to natural cooling is matched. When treatment is paused in the middle, the controller 200 may obtain a time at which the treatment is paused and a time at which the treatment is resumed. The controller 200 may obtain a time during which the treatment is paused. The time during which the treatment is paused may be the time from the time at which the treatment is paused until the time at which the treatment is resumed. When the treatment is paused, the controller 200 may determine the voltage to be applied to the thermoelectric device by applying the time during which the treatment is paused to at least one of the third-fifth table and the third-fifth function. At least one of the third-fifth table and the third-fifth function may be based on the following function.

$$C 4 * t + V 1 = V 2$$

Here, C4 may be a predetermined negative real number that indicates a cooling slope according to natural cooling. V1 may be an initial voltage of the thermoelectric device 330. V2 may be a voltage to be applied to the thermoelectric device 330. t may be the time during which the treatment is paused.

However, the present disclosure is not limited thereto, and at least one of the third-fifth table and the third-fifth function may be based on the following function.

$$C 4 / ( t + a ) + V 1 - C 4 / a = V 2$$

Here, C4 may be a predetermined positive real number that indicates a cooling slope according to natural cooling. t may be the time during which the treatment is paused. a may be a predetermined constant and may be a predetermined positive real number. V1 may be an initial voltage of the thermoelectric device 330. V2 may be a voltage to be applied to the thermoelectric device 330.

V2, which is a voltage to be applied to the thermoelectric device 330, may decrease with an increase in t, which is the time during which the treatment is paused. Also, when the treatment is resumed, the voltage to be applied to the thermoelectric device 330 may increase based on at least one of the third-fourth table, the third-fourth function, the third-fifth table, and the third-fifth function. In this way, since the hand piece maintains an appropriate temperature even when the hand piece does not include a temperature sensor, skin treatment may be performed without damaging the skin surface of a patient. Also, since the hand piece does not include a temperature sensor, the price of the medical skin treatment apparatus may become cheap, and the hand piece may be implemented to be slim, thereby enhancing an aesthetic sense.

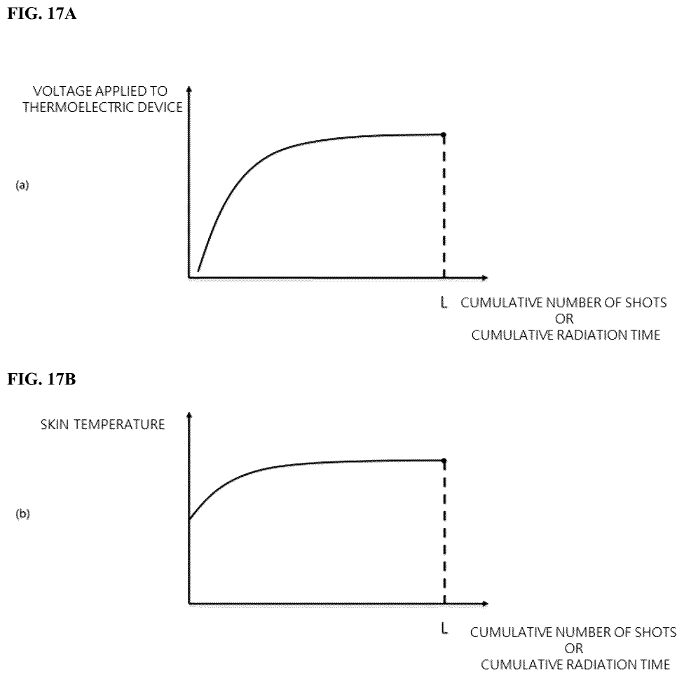

FIGS. 17A and 17B are views for describing a predetermined function according to one embodiment of the present disclosure.

Referring to FIG. 17A, the voltage applied to the thermoelectric device 330 may increase with an increase in the cumulative number of shots and the cumulative radiation time.

Referring to FIG. 17B, the skin temperature may increase with an increase in the cumulative number of shots and the cumulative radiation time. At this time, the skin temperature may be a skin temperature that takes cooling of the thermoelectric device 330 into consideration.

The controller 200 may include a limited number of shots and a limited radiation time. The limited number of shots and the limited radiation time may be predetermined values L of the cumulative number of shots and the cumulative radiation time. From the predetermined values L of the cumulative number of shots and the cumulative radiation time of FIG. 17A, a predetermined voltage may be applied to the thermoelectric device 330. Also, when treatment is continuously performed afterwards, the voltage of the thermoelectric device 330 may be maintained at maximum. Of course, when the treatment is paused, the voltage of the thermoelectric device 330 may decrease.

In the predetermined values L of the cumulative number of shots and the cumulative radiation time of FIG. 17B, the skin temperature of the patient may be a value lower than the maximum value at which the patient may not get burned. When the cumulative number of shots and the cumulative radiation time reach the predetermined values L, heating of the skin surface of the patient and cooling of the thermoelectric device, which are caused by treatment, may form a thermal equilibrium, and the temperature of the skin of the patient may be constantly maintained.

When a problem occurs in the apparatus, and the controller 200 is not able to cool the skin of the patient by adjusting the number of treatment shots, the treatment radiation time, and the voltage applied to the thermoelectric device 330, the controller 200 may output warning sound and warning text through the output part 240 to induce the user to place the hand piece 130 apart from the skin of the patient. Also, the controller 200 may display a time required to sufficiently cool the hand piece and make it possible to provide the number of treatment shots and the treatment radiation time on the output part 240.

The controller 200 may obtain a treatment purpose based on an input of a user. The treatment purpose may include at least one of a treatment site and information on a treatment type. The controller 200 may automatically provide at least one of the number of ultrasound treatment shots and the treatment radiation time that correspond to the treatment purpose.

For example, the number of treatment shots and the treatment radiation time may vary according to the treatment site. The treatment site may include the skin of at least one of the face, arm, leg, and abdomen of the patient. The skin thickness may vary according to the treatment site. The controller 200 may obtain a skin thickness value prestored according to the treatment site. The controller 200 may obtain at least one of the number of treatment shots and the treatment radiation time prestored according to the skin thickness value.

According to various embodiments of the present disclosure, the medical skin treatment apparatus 100 may include a sensor measuring a skin thickness. The controller 200 may control the number of treatment shots and the treatment radiation time using a skin thickness value measured using the sensor.

The medical skin treatment apparatus 100 may measure a skin thickness using the sensor measuring a skin thickness. The number of treatment shots and the treatment radiation time may increase with an increase in the skin thickness measured by the sensor. The number of treatment shots and the treatment radiation time may decrease with a decrease in the measured skin thickness.

Also, the number of treatment shots and the treatment radiation time may vary according to the treatment type. The treatment type may include information on a skin layer where focal points will be formed according to the treatment purpose such as wrinkle treatment and fat removal. The number of treatment shots and the treatment radiation time may increase with an increase in the depth of the skin layer where the focal points will be formed. The number of treatment shots and the treatment radiation time may decrease with a decrease in the depth of skin layer.

At least one of the first table, the first function, the second table, the second function, the third table, and the third function may be different for at least one of a frequency of a transducer, the number of formed focal points, and the treatment purpose. Also, at least one of the first function, the second function, and the third function may be different for at least one of a frequency of a transducer, the number of formed focal points, and the treatment purpose. For example, the skin temperature may rapidly increase with an increase in the frequency of the transducer, an increase in the number of formed focal points, and an increase in the depth of the skin layer that corresponds to the treatment purpose. With an increase in the skin temperature, the number of treatment shots and the treatment radiation time that may be provided by the hand piece 130 may decrease, and the voltage applied to the thermoelectric device 330 may increase. Therefore, at least one of the number of treatment shots and the treatment radiation time obtained through at least one of the first table, the first function, the second table, and the second function may decrease with an increase in the frequency of the transducer, an increase in the number of formed focal points, and an increase in the depth of the skin layer that corresponds to the treatment purpose. The voltage applied to the thermoelectric device 330 that is obtained through the third table may increase with an increase in the frequency of the transducer, an increase in the number of formed focal points, and an increase in the depth of the skin layer that corresponds to the treatment purpose.

Although cooling of the hand piece of the medical skin treatment apparatus using ultrasonic waves has been described above, the present disclosure is not limited thereto. The medical skin treatment apparatus may use at least one of a function and a table to cool the hand piece of the medical skin treatment apparatus 100 using a laser. The medical skin treatment apparatus 100 using a laser may not only use a thermoelectric device but also eject a cooling gas. Since a process of cooling a surface at which the hand piece and the skin of the patient come into contact using a thermoelectric device has been described above, overlapping description thereof will be omitted.

When a temperature of the surface at which the skin of the patient and the hand piece come into contact is a predetermined temperature or more, the medical skin treatment apparatus 100 using ultrasonic waves or a laser may eject a cooling gas to lower the skin temperature. In relation to this, since the medical skin treatment apparatus 100 of the present disclosure does not include a temperature sensor, it is necessary to determine a point in time at which the cooling gas is ejected.

Typically, the medical skin treatment apparatus 100 may use at least one of the first table, the first function, the second table, and the second function to determine the point in time at which the cooling gas is ejected.

More specifically, the controller 200 may predict a temperature of a contact surface between the skin of the patient and the hand piece (skin temperature). The skin temperature may increase with an increase in the cumulative number of shots and the cumulative radiation time of the hand piece. The controller 200 may determine the skin temperature based on at least one of the cumulative number of shots and the cumulative radiation time using at least one of the first table, the first function, the second table, and the second function. The controller 200 may determine a skin temperature (V0) by applying the cumulative number of shots obtained in Step (910) of FIG. 9 and the cumulative radiation time obtained in Step (920) of FIG. 9 to at least one of the first table, the first function, the second table, and the second function. Also, when the skin temperature is a predetermined critical temperature or more, the controller 200 may eject a cooling gas of a predetermined amount. Also, the controller 200 may determine a temperature after ejection of the cooling gas based on the skin temperature and the predetermined amount of the cooling gas. The temperature after ejection of the cooling gas may become lower than the skin temperature with an increase in the predetermined amount of the cooling gas and may become significantly lower than the skin temperature with an increase in a difference between the current skin temperature and a predetermined temperature of the cooling gas.

For example, the temperature after ejection may be determined by the following equation.

Temperature after ejection=(k*skin temperature+m*amount of cooling gas*temperature of cooling gas)/(k+m*amount of cooling gas)

Here, k and m may be predetermined real numbers. k may be a predetermined value relating to a heat capacity of the skin or hand piece, and m may be a predetermined value relating to specific heat of the cooling gas.

The controller 200 may determine the skin temperature (V1) again by setting the temperature after ejection as T0 of at least one of the first table, the first function, the second table, and the second function.

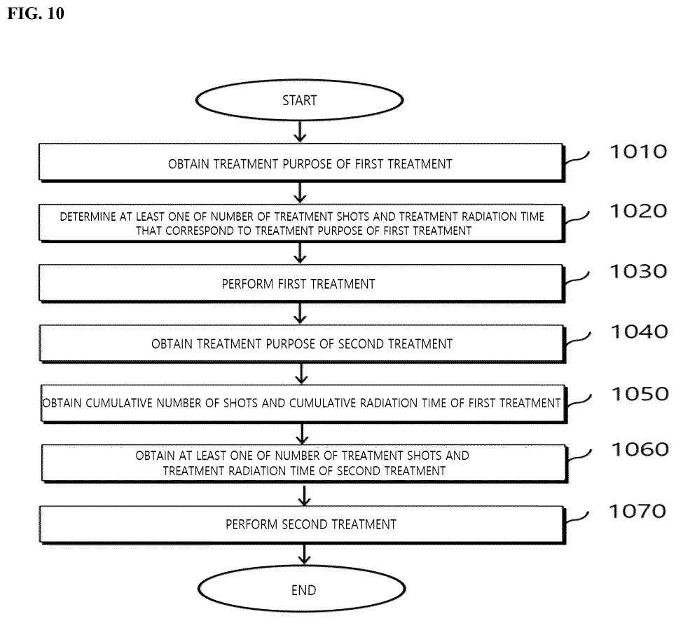

FIG. 10 is a flowchart for describing an operation of the medical skin treatment apparatus according to one embodiment of the present disclosure.

According to one embodiment of the present disclosure, a first treatment may start when the power of the medical skin treatment apparatus 100 is turned on. The controller 200 may perform obtaining a treatment purpose of the first treatment (1010). The controller 200 may perform determining at least one of the number of treatment shots and the treatment radiation time that correspond to the treatment purpose of the first treatment (1020). The controller 200 may automatically set start values of the number of treatment shots and the treatment radiation time according to the treatment purpose. In addition, values input by a user may be used as the start values of the number of treatment shots and the treatment radiation time. The medical skin treatment apparatus 100 may perform performing the first treatment (1030). The plurality of transducers 510 and 520 may emit vibration energy based on the set number of treatment shots and treatment radiation time. A voltage may be applied to the thermoelectric device 330 based on the set number of treatment shots and treatment radiation time, and the cooling surface of the thermoelectric device 330 may be cooled. When the treatment according to the set number of treatment shots and treatment radiation time is all done or the first treatment is forcibly ended by the user, the controller 200 may perform a second treatment. The second treatment may be a separate treatment including the same treatment purpose as the first treatment or including a different treatment purpose. The controller 200 may perform obtaining a treatment purpose of the second treatment (1040). The controller 200 may receive the treatment purpose of the second treatment. The controller 200 may perform obtaining at least one of a cumulative number of shots and a cumulative radiation time of the first treatment (1050). When the treatment purpose of the second treatment is received, the controller 200 may obtain a time from a time at which the first treatment has ended until a time at which the treatment purpose of the second treatment is received as a time during which treatment is paused. The controller 200 may perform the steps of FIGS. 7 to 9 using at least one of the cumulative number of shots of the first treatment, the cumulative radiation time of the first treatment, and the time during which treatment is paused. The controller 200 may perform obtaining at least one of a number of treatment shots and a treatment radiation time of the second treatment (1060). The controller 200 may determine at least one of the number of treatment shots and the treatment radiation time relating to the second treatment. The controller 200 may determine a voltage applied to the thermoelectric device 330 in relation to at least one of the number of treatment shots and the treatment radiation time of the second treatment. The controller 200 may perform performing the second treatment (1070). The plurality of transducers 510 and 520 may emit vibration energy based on the determined number of treatment shots and treatment radiation time. The cooling surface of the thermoelectric device 330 may be cooled based on the determined voltage.

A patient may feel hotness or pain during treatment due to an error in the number of treatment shots and treatment radiation time or skin differences between individual patients. When the hand piece 130 is detached from the skin of a patient, the controller 200 may determine that treatment is paused for a moment. The controller 200 may automatically readjust the number of treatment shots and the treatment radiation time or may receive the number of treatment shots and the treatment radiation time from a user. The controller 200 may obtain a cumulative number of shots and a cumulative radiation time based on the changed number of treatment shots and treatment radiation time. The newly-obtained cumulative number of shots and cumulative radiation time may be applied to the number of treatment shots and the treatment radiation time that are subsequently provided.

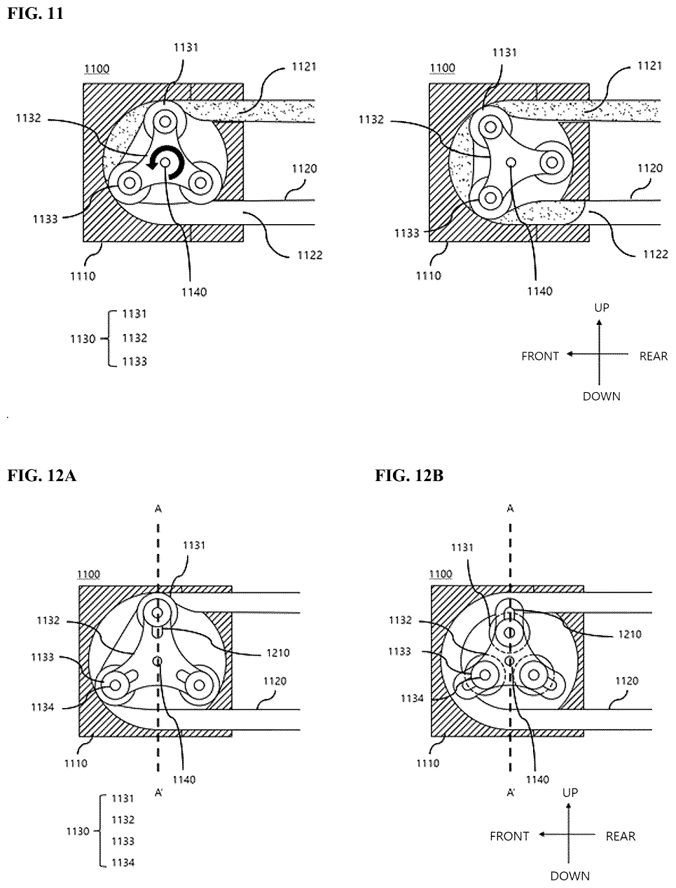

FIG. 11 is a view illustrating a drug supply according to one embodiment of the present disclosure.

The drug supply 1100 may include a pressure housing 1110 in which an inlet 1121 coupled to the drug reservoir and an outlet 1122 coupled to the drug injection part are formed. The inlet 1121 and the outlet 1122 may be formed in the same surface on one side of the pressure housing 1110.

The drug supply 1100 may include a rubber tube 1120 located inside the pressure housing 1110, coupling the inlet 1121 and the outlet 1122, and conveying a drug.

The drug supply 1100 may be located inside the pressure housing 1110. The drug supply 1100 may be coupled to be rotatable relative to the pressure housing 1110 about a driving shaft 1140 . . . . The drug supply 1100 may include a rotator 1130 including a plurality of rollers 1131, a rotary plate 1132, and a roller support part 1133. The rotator 1130 may include the rotary plate 1132, the roller support part 1133, and the plurality of rollers 1131. The rotary plate 1132 may have protrusions formed thereon at one of 72°, 90°, 120°, and 180° intervals about the driving shaft 1140. However, the present disclosure is not limited thereto, and an angle between the protrusions may be any other predetermined angle. The roller support part 1133 may be coupled to the protrusions of the rotary plate 1132 and coupled to a rotating shaft 1134 of one of the plurality of rollers 1131. One of the plurality of rollers 1131 may be coupled to the roller support part 1133. One roller 1131 may be coupled one-to-one to one roller support part 1133.

The drug supply 1100 may include the driving shaft 1140 passing through a center of the rotator 1130 and may include a driver rotating the rotator 1130 about the driving shaft 1140. The driver may include a motor. The driving shaft 1140 may be coupled to the driver.

At least one surface inside the pressure housing 1110 may include a curved surface. More specifically, a surface opposite to the surface where the inlet 1121 and the outlet 1122 are formed may be a curved surface having a circular arc. At least one of the plurality of rollers included in the rotator may come into contact with the curved surface inside the pressure housing 1110 and press the rubber tube. When the rotator rotates, the roller may rotate along the curved surface inside the pressure housing.

As the pressure housing 1110 and the plurality of rollers 1131 press the rubber tube 1120, a space may be formed inside the rubber tube 1120 between the pressure housing 1110 and the plurality of rollers 1131 in the drug supply 1100. A drug may be included in the space. In the drug supply 1100, as the rotator 1130 rotates, the pressure housing 1110 and the plurality of rollers 1131 rotate while pressing the rubber tube 1120 conveying the drug, and thus the drug in the rubber tube 1120 may also rotate while separated. The separated drug may be conveyed to the skin of a patient through the rubber tube 1120. The amount of drug to be injected may be predetermined for each treatment type of the medical skin treatment apparatus 100. The controller 200 may store angles of rotation of the drug supply 1100 that correspond to predetermined amounts of the drug. The predetermined amount of the drug may increase with an increase in the angle of rotation of the drug supply 1100. The drug supply 1100 may supply the drug of a predetermined amount to the patient by the rotator 1130 rotating at a predetermined angle of rotation. The drug supply 1100 may accurately supply different amounts of the drug to the skin of a patient for each type of ultrasound treatment.

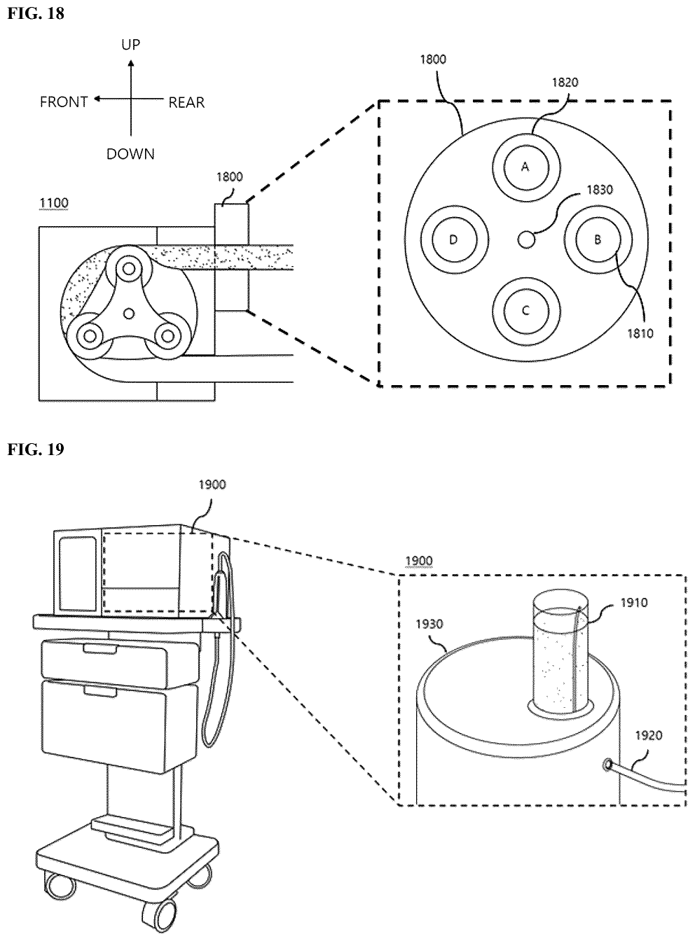

FIG. 18 is a view illustrating the drug supply according to one embodiment of the present disclosure.

According to various embodiments of the present disclosure, the drug reservoir may store a plurality of drugs different from each other. The plurality of drugs may have a separate rubber tube 1120 for each drug. Referring to FIG. 18, the drug supply 1100 may include a rubber tube fixing part 1800 having a circular shape. The rubber tube fixing part 1800 may fix the plurality of rubber tubes 1120. The plurality of rubber tubes 1120 may be arranged in a circular shape about a fixing part rotating shaft 1830 on the rubber tube fixing part 1800.

The rubber tube fixing part 1800 may include a plurality of fixing parts 1810 dividing the rubber tubes 1120 for each drug and fixing the rubber tubes 1120. For example, when drugs A, B, C, and D are stored in the drug reservoir, the rubber tube fixing part 1800 may include fixing parts 1810 each corresponding to one of the drugs A, B, C, and D. Therefore, the rubber tubes coupled to the drugs A, B, C, and D may be fixed to the fixing parts 1810 of the rubber tube fixing part 1800 that correspond to the drugs A, B, C, and D.

The rubber tube coupled to the drug reservoir and the drug supply 1100 and the rubber tube coupled to the drug supply 1100 and the drug injection part 410 may be separated or coupled based on the inlet of the drug supply 1100. The rubber tube fixing part 1800 may include a plurality of coupling parts 1820 through which the plurality of rubber tubes 1120 may be coupled to an inlet of the drug reservoir. The rubber tube fixing part 1800 may include the fixing part rotating shaft 1830. The rubber tube fixing part 1800 may rotate relative to the drug supply 1100 about the fixing part rotating shaft 1830. The inlet 1121 of the drug supply 1100 may be coupled to one of the plurality of rubber tubes 1120 different from each other by the coupling part 1820 of the rubber tube fixing part 1800.

The medical skin treatment apparatus 100 may receive information on each of the plurality of drugs stored in the drug reservoir from the user through the input part 250. The controller 200 may store different drug information for each type of treatment that may be performed by the medical skin treatment apparatus 100.

When the user starts treatment, the controller 200 may obtain drug information relating to the type of the treatment. The controller 200 may start supply of a drug corresponding to the drug information from the drug reservoir. The controller 200 may rotate the rubber tube fixing part 1800 and couple the rubber tube corresponding to the drug information to the drug supply 1100. Therefore, the drug supply 1100 may appropriately provide a drug suitable for each type of treatment.

FIGS. 12A and 12B are views illustrating an operation of the drug supply according to one embodiment of the present disclosure.

In the drug supply 1100, when the medical skin treatment apparatus is deactivated, the plurality of rollers 1131 may be stored inside the rotary plate 1132 and may not apply pressure to the rubber tubes 1120. The controller 200 may determine deactivation of the medical skin treatment apparatus 100. When a request to turn off the power of the medical skin treatment apparatus 100 is received from the user, the controller 200 may determine that the medical skin treatment apparatus 100 has been deactivated. Also, when the medical skin treatment apparatus 100 does not operate for a predetermined amount of time, the controller 200 may determine that the medical skin treatment apparatus 100 has been deactivated.

When the medical skin treatment apparatus is activated, the plurality of rollers 1131 may protrude to the outside of the rotary plate 1132, and the pressure housing 1110 and the rollers 1131 may apply pressure to the rubber tubes 1120. The controller 200 may determine activation of the medical skin treatment apparatus 100. When a new operation is input by the user while the medical skin treatment apparatus 100 is in a deactivated state, the controller 200 may determine that the medical skin treatment apparatus 100 has been activated again.

A plurality of movement holes 1210 may each be formed in one of the plurality of protrusions of the rotary plate 1132. A single roller 1131, a single roller support part 1133, and a single rotating shaft 1134 may be coupled to each of the movement holes 1210.

A structure in which the roller protrudes from the rotary plate or is stored inside the rotary plate will be described below.

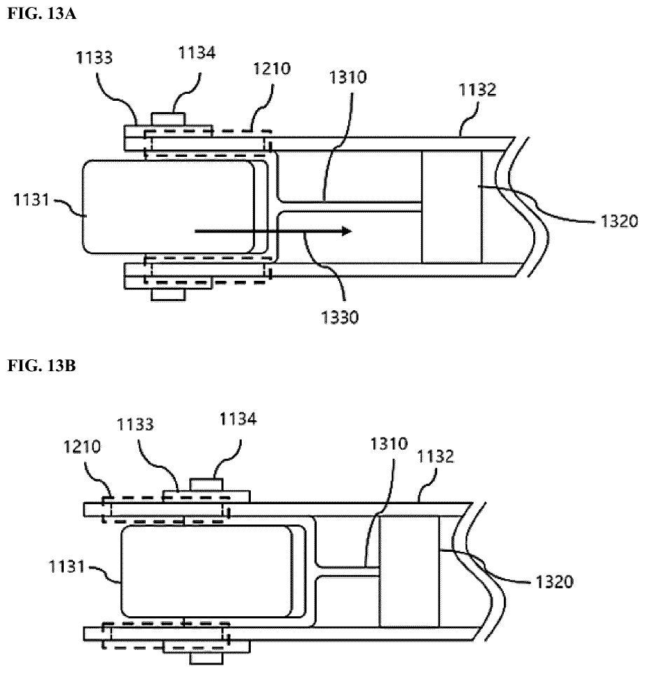

FIGS. 13A and 13B are views for describing an operation of a rotator according to one embodiment of the present disclosure.

FIGS. 13A and 13B show cross-sections along line A-A′ of FIGS. 12A and 12B. Referring to FIGS. 12A, 12B, 13A and 13B, the rotator 1130 may include a connecting rod 1310 and a storage driver 1320. The connecting rod 1310 may connect the rotating shaft 1134 and the storage driver 1320. Due to the storage driver 1320, the rotating shaft 1134 may move in a first direction 1330 and a direction opposite to the first direction 1330 along the movement hole 1210. The first direction 1330 is a direction toward a center of the rotary plate 1132 and may be a direction in which the roller 1131 is stored or a direction toward the driving shaft 1140. The direction opposite to the first direction 1330 may be a direction in which the roller 1131 protrudes to the outside of the rotary plate 1132.

Referring to FIG. 13A, the roller 1131 may be located outside the rotary plate 1132. At this time, the roller 1131 may be in a state in which it is in close contact with the pressure housing 1110 and pressing the rubber tube 1120. When the medical skin treatment apparatus 100 is deactivated, the controller 200 may control the storage driver 1320. The storage driver 1320 may include a motor. The storage driver 1320 may include a rack coupled to the motor and a pinion coupled to the connecting rod 1310. The connecting rod 1310 may linearly reciprocate through an operation of the motor of the storage driver 1320. Therefore, due to the storage driver 1320, the rotating shaft 1134 may move in the first direction 1330 along the movement hole 1210. A length of the movement hole 1210 may be greater than or equal to a diameter of the rubber tube 1120.

Referring to FIG. 13B, the roller 1131 may be in a state in which it is stored inside the rotary plate 1132 due to the rotating shaft 1134 moving in the first direction 1330 along the movement hole 1210. At this time, the roller 1131 may not come into close contact with the pressure housing 1110 or at least not apply pressure to the rubber tube 1120. Since the rubber tube 1120 is not pressed or stretched while the medical skin treatment apparatus is in a deactivated state, the service life of the rubber tube may increase.

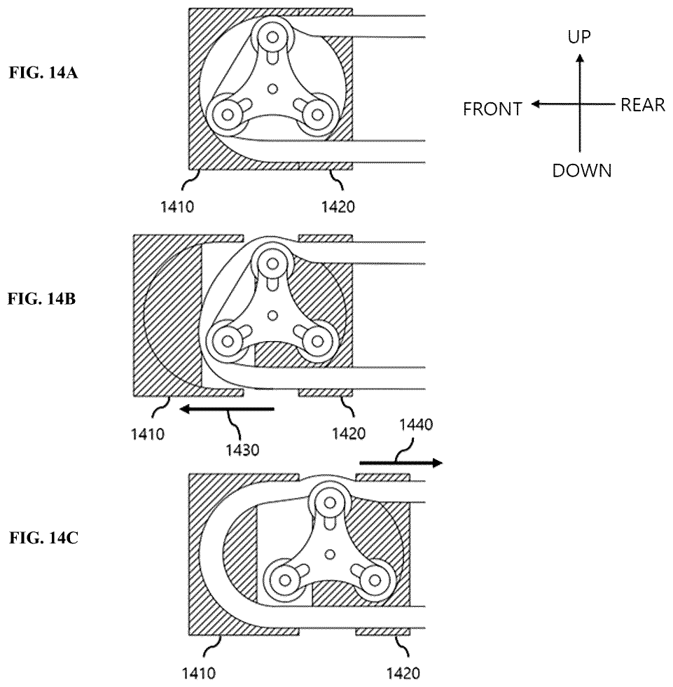

FIGS. 14A, 14B, 14C are views illustrating an operation of the drug supply according to one embodiment of the present disclosure.

Referring to FIG. 14A, the drug supply 1100 may be in a state in which the pressure housing 1110 and the roller 1131 are pressing the rubber tube. The pressure housing 1110 of the drug supply 1100 may include a pressure housing moving part 1410 and a pressure housing base part 1420. At least one of the main body 110 and the pressure housing 1110 of the medical skin treatment apparatus 100 may include a housing driver. The housing driver may be coupled to at least one of the pressure housing moving part 1410 and the pressure housing base part 1420. The housing driver may move each of the pressure housing moving part 1410 and the pressure housing base part 1420 in a front-rear direction.

Referring to FIG. 14B, in the drug supply 1100, when the medical skin treatment apparatus 100 is deactivated, the pressure housing moving part 1410 included in the pressure housing 1110 may move forward (1430) relative to the pressure housing base part 1420 included in the pressure housing 1110 due to the housing driver and may not apply pressure to the rubber tube. That is, the pressure housing 1110 may become distant from the roller included in the rotator 1130 and not apply pressure to the rubber tube.

When the medical skin treatment apparatus 100 is activated, the pressure housing moving part 1410 may move backward (1440) relative to the pressure housing base part 1420 due to the housing driver and may apply pressure to the rubber tube. At this time, the pressure housing moving part 1410 may be coupled to the pressure housing base part 1420 again as illustrated in FIG. 14A.

Referring to FIG. 14C, in the drug supply 1100, when the medical skin treatment apparatus 100 is deactivated, the rotator 1130 may move backward (1440) and may not apply pressure to the rubber tube. The pressure housing base part 1420 may be coupled to the rotator 1130 by the driving shaft 1140 of the rotator 1130. By the pressure housing base part 1420 moving backward (1440) due to the housing driver, the roller included in the rotator 1130 may become distant from the rubber tube and may not apply pressure thereto.

When the medical skin treatment apparatus 100 is activated, the rotator 1130 may move forward (1430) and may apply pressure to the rubber tube. By the pressure housing base part 1420 moving forward (1430) again due to the housing driver, the pressure housing base part may be coupled to the pressure housing moving part 1410 as illustrated in FIG. 14A. Therefore, the roller included in the rotator 1130 may apply pressure to the rubber tube again.

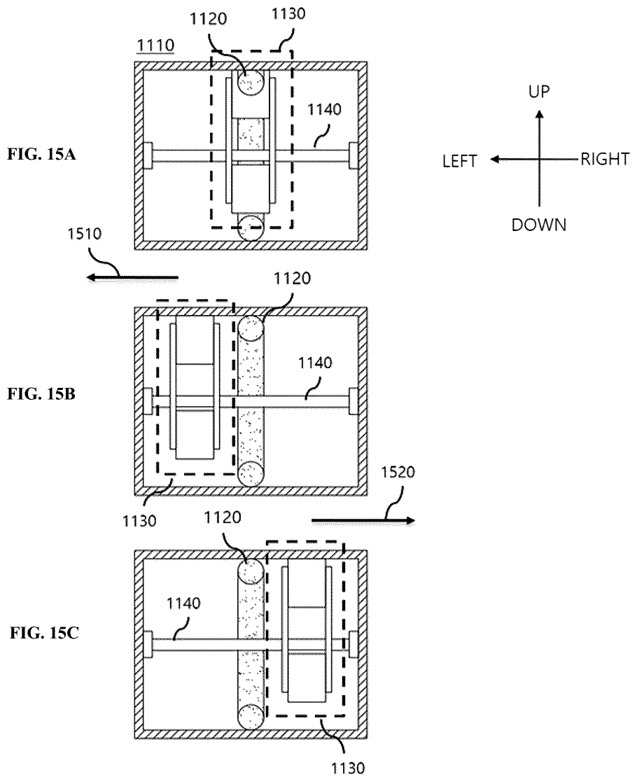

FIGS. 15A, 15B, 15C are views illustrating an operation of the drug supply according to one embodiment of the present disclosure.

Referring to FIG. 15A, the drug supply 1100 may be in a state in which the pressure housing 1110 and the roller 1131 are pressing the rubber tube 1120. The pressure housing 1110 may include a space that is expanded to the left and right and is sufficient for accommodating the rotator. The driving shaft 1140 of the rotator 1130 may pass through the space inside the pressure housing 1110. At least one of the main body 110 and the rotator 1130 of the medical skin treatment apparatus 100 may include a rotator driver. The rotator driver may move the rotator 1130 in a left-right direction along the driving shaft 1140 of the rotator 1130.

Referring to FIG. 15B, in the drug supply 1100, when the medical skin treatment apparatus 100 is deactivated, the rotator may move leftward (1510) and may not apply pressure to the rubber tube. When the medical skin treatment apparatus 100 is activated, the rotator 1130 may move rightward (1520) and may apply pressure to the rubber tube again as illustrated in FIG. 15A.

Referring to FIG. 15C, in the drug supply 1100, when the medical skin treatment apparatus 100 is deactivated, the rotator may move rightward (1520) and may not apply pressure to the rubber tube. When the medical skin treatment apparatus 100 is activated, the rotator 1130 may move leftward (1510) and may apply pressure to the rubber tube again as illustrated in FIG. 15A.

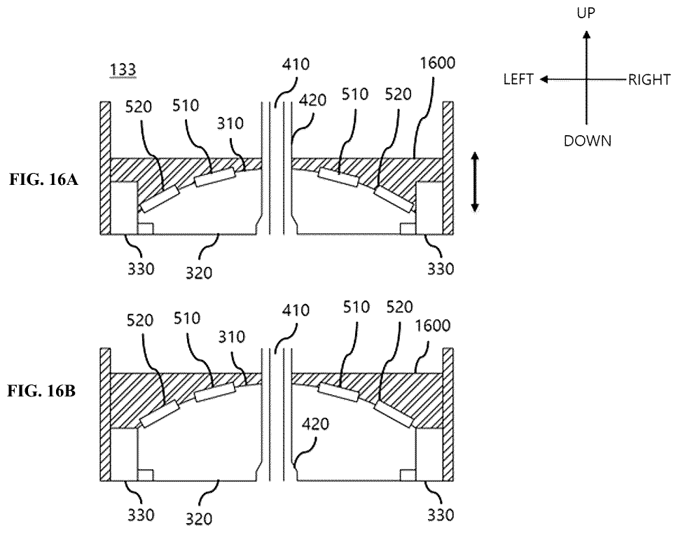

FIGS. 16A and 16B are views illustrating the base part according to one embodiment of the present disclosure.

The medical skin treatment apparatus 100 may vertically move the transducer arrangement surface 310 and change the depths of focal points of the transducers 510 and 520 based on a depth of focal points input by the user. The base part 133 may include an arrangement surface driver 1600. The arrangement surface driver 1600 may include a motor. The arrangement surface driver 1600 may include a rack coupled to the motor and a pinion formed on the transducer arrangement surface 310. The transducer arrangement surface 310 may vertically and linearly reciprocate through an operation of the motor of the arrangement surface driver 1600. Therefore, the arrangement surface driver 1600 may vertically move the transducer arrangement surface 310 along a wall surface of the base part 133 and the skin suction part 420.

The controller 200 of the medical skin treatment apparatus 100 may obtain a focal point depth of at least one of the plurality of transducers 510 and 520 based on an input of a user. The controller 200 may store focal point depth values that corresponding to different treatment purposes. When the user has not input a focal point depth, the controller 200 may automatically obtain a focal point depth that corresponds to a treatment purpose. The controller 200 may store focal point ranges according to positions of the transducer arrangement surface 310.

When a focal point depth deviates from a focal point range according to the current position of the transducer arrangement surface 310, the transducer arrangement surface 310 may vertically move to move the focal points of the plurality of transducers 510 and 520.

When a focal point depth set by the user is below the focal point range according to the current position of the transducer arrangement surface 310, the transducer arrangement surface 310 may move upward. By the transducer arrangement surface 310 moving upward, the plurality of transducers 510 and 520 may move together, the depths of the focal points formed by the plurality of transducers 510 and 520 may decrease, and treatment may be performed on a shallower site.

When a focal point depth set by the user is beyond the focal point range according to the current position of the transducer arrangement surface 310, the transducer arrangement surface 310 may move downward. By the transducer arrangement surface 310 moving downward, the plurality of transducers 510 and 520 may move together, the depths of the focal points formed by the plurality of transducers 510 and 520 may increase, and treatment may be performed on a deeper site.

When a focal point depth set by the user deviates from a focal point range that may be reached by movement of the transducer arrangement surface 310, the output part 240 may be controlled to output warning sound and a warning message that recommend the focal point depth to be reset.

FIG. 19 is a view illustrating a drug reservoir 1900 according to one embodiment of the present disclosure.

The medical skin treatment apparatus 100 may include the drug reservoir 1900 storing a drug to be injected into the skin of a patient.

The drug reservoir 1900 may include a drug container 1910. The drug container 1910 may include the drug case described above with reference to FIG. 1. The drug container 1910 may include a bottle body 1912 having a cylindrical shape and a bottle neck 1911 having a cylindrical shape and having an opening formed therein. A diameter of the bottle neck 1911 of the drug container 1910 may be smaller than a diameter of the bottle body 1912. The present disclosure is not limited thereto, and the diameter of the bottle neck 1911 of the drug container 1910 may be equal to the diameter of the bottle body 1912. In one embodiment of the present disclosure, during the operation of the medical skin treatment apparatus 100, the bottle neck 1911 of the drug container 1910 may face downward, and the bottle body 1912 may face upward as illustrated in FIG. 19.

The drug reservoir 1900 may include a drug hose 1920. The drug hose 1920 may include the rubber tubes 132 and 1120 described above. The drug hose 1920 may be made of a corrosion-resistant material that is not corroded by drugs. The drug hose 1920 may couple the drug reservoir 1900 and the hand piece 130 and convey a drug.

The drug reservoir 1900 may include a pump module 1930. The pump module 1930 may suction a drug at a high pressure from the drug container 1910 and discharge the drug to the drug hose 1920 coupled to the hand piece 130. The present disclosure is not limited thereto, and the drug inside the drug container 1910 may be naturally supplied to the pump module 1930 without being suctioned at a high pressure. The drug container 1910 may be coupled to the pump module 1930 with the opening of the drug container 1910 facing downward to allow the drug inside the drug container 1910 to be supplied to the pump module 1930. The drug may be supplied to the pump module 1930 due to gravity, and the pump module 1930 may not generate a pressure for suctioning the drug from the drug container 1910. The pump module 1930 may deliver the supplied drug to the hand piece 130.

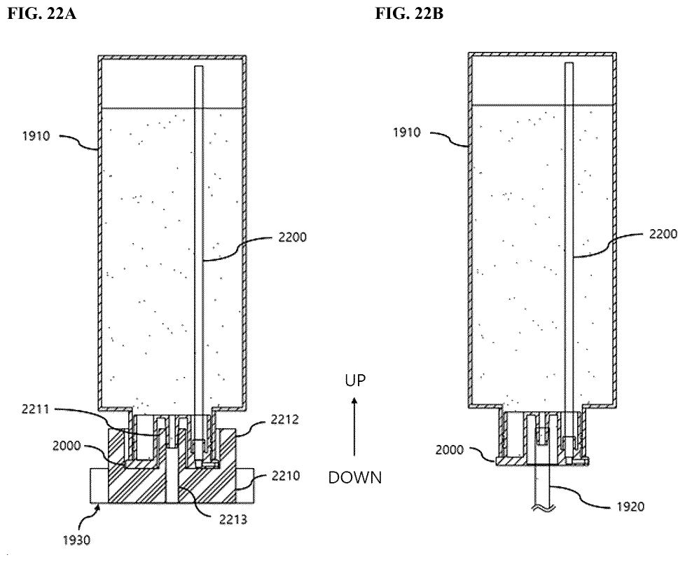

The pump module 1930 may be directly coupled to the drug container 1910 through a container coupling part 2210 or may be coupled to the drug container 1910 through a separate drug hose 1920. The coupling between the pump module 1930 and the drug container 1910 will be described below with reference to FIG. 22.

The pump module 1930 may move the drug inside the drug container 1910 to the hand piece 130 through the drug hose 1920. The pump module 1930 may include a pump power part. The pump power part may include at least one of a compressor, an engine, and a motor and may supply power to the pump module 1930. The pump module 1930 may move the drug at a high pressure using the pump power part. The controller 200 may control the pump module 1930 to adjust the amount of supplied drug.

The drug reservoir 1900 may include a stopper part 2000. The stopper part 2000 may be inserted into the opening of the drug container 1910 to prevent leakage of the drug. The stopper part 2000 may have a duct 2150 through which the drug hose 1920 passes formed therein. The stopper part 2000 may be formed of the same material as a whole. For example, the stopper part 2000 may be formed of a plastic or silicone material. However, the present disclosure is not limited thereto, and the stopper part 2000 may be formed of various other materials that can block leakage of the drug inside the drug container 1910. Also, the stopper part 2000 may be partially formed of different materials

The drug container 1910 may be coupled to the pump module 1930 in a state in which the drug container 1910 and the stopper part 2000 are coupled to each other. The stopper part 2000 may include a structure for coupling to the pump module 1930.

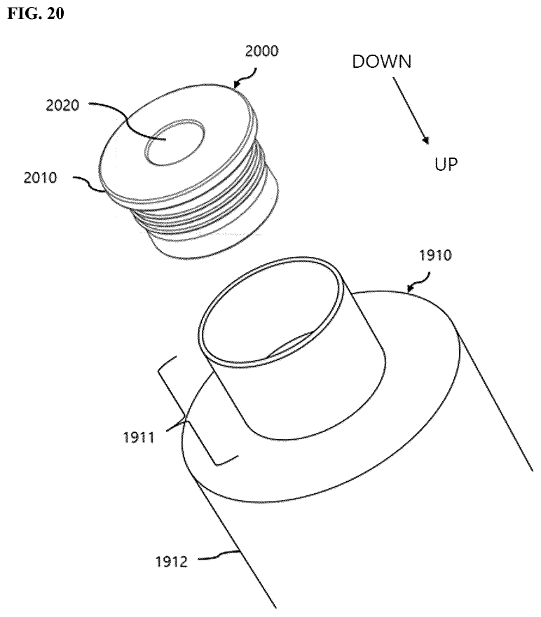

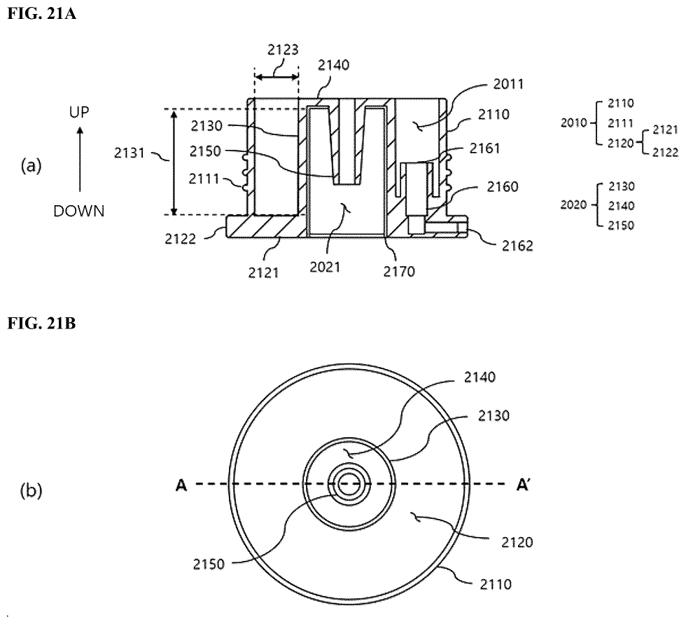

The shape and detailed structure of the stopper part 2000 will be described below with reference to FIGS. 20 and 21.

FIGS. 20, 21A and 21B are views illustrating a stopper part according to one embodiment of the present disclosure.

FIG. 20 illustrates the overall shape of the stopper part 2000. FIG. 21A illustrates a cross-sectional view along line A-A′ of the stopper part 2000 of FIG. 21B, and FIG. 21B illustrates a bottom view of the stopper part 2000.

The stopper part 2000 may include an outer body 2010 and an inner body 2020. The outer body 2010 and the inner body 2020 may be integrally formed and form a concave or convex shape.

The stopper part 2000 may include the outer body 2010. The outer body 2010 may have at least a portion coming into contact with an inner circumferential surface of the opening of the drug container 1910. The outer body 2010 may include an outer wall 2110. The outer wall 2110 may have a cylindrical shape that has at least one of an open upper side and an open lower side and that vertically extends. The outer body 2010 may include a stopper part closing surface 2120 having a ring shape. The stopper part closing surface may be a ring-shaped plate. The stopper part closing surface 2120 may be integrally formed with the outer wall 2110. The stopper part closing surface 2120 may include a lower closing surface 2121. The lower closing surface 2121 may be formed in a ring shape. The lower side of the outer wall 2110 may be at least partially closed by the lower closing surface 2121 having a ring shape. The lower closing surface 2121 may be perpendicular to the outer wall 2110. An upper end of the lower closing surface 2121 may be coupled to a lower end of the outer wall 2110. At this time, a radius of an outer circumferential surface of the lower closing surface 2121 may be equal to a radius of an outer circumferential surface of the outer wall 2110. The drug inside the drug container 1910 may be blocked by the lower closing surface 2121 and may be discharged only through the duct 2150.

The stopper part closing surface 2120 may include a catching part 2122. The catching part 2122 may be formed to extend in a radial direction of the lower closing surface 2121. The radial direction of the lower closing surface 2121 may be a direction expanding outward from a center of the lower closing surface 2121. A radius of the catching part 2122 may be greater than a radius of the outer wall 2110. When the stopper part 2000 is inserted, an upper end of the catching part 2122 may come into contact with a lower end of the bottle neck 1911 of the drug container 1910. That is, the catching part 2122 may be placed across the bottle neck 1911 forming the opening of the drug container 1910 without being inserted into the opening of the drug container 1910. Therefore, the catching part 2122 may prevent the outer body 2010 from being completely inserted into the drug container 1910.

In one embodiment of the present disclosure, the outer body 2010 may include at least one stopper protrusion 2111. The stopper protrusion 2111 may be formed to be convex along the outer circumferential surface of the outer wall 2110. The stopper protrusion 2111 may extend in the radial direction of the outer wall 2110.

The stopper protrusion 2111 may protrude in a direction parallel to the stopper part closing surface 2120. A radius of the stopper protrusion 2111 may be greater than or equal to a radius of an inner circumferential surface of the bottle neck of the drug container 1910 to allow the stopper protrusion 2111 to come into contact with the inner circumferential surface of the bottle neck of the drug container 1910. Therefore, when the stopper part 2000 is inserted into the drug container 1910, the stopper protrusion 2111 may allow the stopper part 2000 and the drug container 1910 to tightly come into close contact with each other. By allowing the stopper part 2000 and the drug container 1910 to come into close contact with each other, the stopper protrusion 2111 may prevent the drug from leaking between the outer wall 2110 and the drug container 1910. Also, a plurality of stopper protrusions 2111 may be formed on the outer body 2010. For example, three stopper protrusions 2111 may be formed on the outer body 2010 as illustrated in FIG. 21A. By the plurality of stopper protrusions 2111 being formed, even when one stopper protrusion 2111 does not properly come into close contact with the drug container 1910, the other stopper protrusions 2111 may come into close contact with the drug container 1910 and prevent leakage of the drug.

In another embodiment of the present disclosure, the stopper protrusion 2111 may be formed diagonally relative to the stopper part closing surface 2120 and may serve as screw threads. Screw threads that correspond to the stopper protrusion 2111 may be formed on the inner circumferential surface of the bottle neck of the drug container 1910. Screw coupling between the drug container 1910 and the outer body 2010 may allow the stopper part 2000 and the drug container 1910 to tightly come into close contact with each other.

At least one stopper protrusion 2111 may be formed of a material that is different from a material of the outer body 2010. For example, at least one stopper protrusion 2111 may be formed of a synthetic material with good elasticity and strong friction, such as silicone or rubber, and may increase adhesion between the stopper part 2000 and the drug container 1910. However, the present disclosure is not limited thereto, and the stopper protrusion 2111 may be formed of the same material as the outer body 2010.

The stopper part 2000 may include the inner body 2020. The inner body 2020 may include an inner wall 2130. The inner wall 2130 may have a cylindrical shape that has an open upper side and an open lower side and vertically extends. The inner wall 2130 having the cylindrical shape may be coupled to be perpendicular to the lower closing surface 2121. Specifically, a lower end of the inner wall 2130 may be coupled to an upper end of the lower closing surface 2121. A radius of an inner circumferential surface of the inner wall 2130 may be equal to a radius of an inner circumferential surface of the lower closing surface 2121. The inner wall 2130 may support the stopper part 2000 to prevent the stopper part 2000 from deviating from the pump module 1930 when the stopper part 2000 and the pump module 1930 are coupled to each other.

The inner body 2020 may include an upper closing surface 2140. The upper closing surface 2140 may be integrally formed with the inner wall 2130. The upper closing surface 2140 may be formed in a ring shape. An upper side of the inner wall 2130 may be at least partially closed by the upper closing surface 2140 having a ring shape. The upper closing surface 2140 may be perpendicular to the inner wall 2130. A lower end of the upper closing surface 2140 may be coupled to an upper end of the inner wall 2130. A radius of an outer circumferential surface of the upper closing surface 2140 may be equal to a radius of an outer circumferential surface of the inner wall 2130. The drug inside the drug container 1910 may be blocked by the upper closing surface 2140 and may be discharged only through the duct 2150.

According to various embodiments of the present disclosure, the upper closing surface 2140 may be formed to be inclined toward the duct 2150 as illustrated in FIG. 24.

FIG. 24 is a cross-sectional view of the stopper part according to one embodiment of the present disclosure.

Referring to FIG. 24 for a moment, the upper closing surface 2140 may include a slope that is tilted relative to a surface perpendicular to the inner wall 2130. Accordingly, the duct 2150 may be formed at a lower position when the upper closing surface 2140 is formed perpendicular to inner wall 2130. The upper closing surface 2140 may be a surface coming into contact with the drug inside the drug container 1910. The upper closing surface 2140 may be formed to be inclined toward the duct 2150 to allow the drug to flow to the duct 2150. That is, the upper closing surface 2140 may allow the drug inside the drug container 1910 to be more smoothly discharged through the duct 2150. At this time, the slopes of inclinations of an upper surface and a lower surface of the upper closing surface 2140 may be equal to each other. However, the present disclosure is not limited thereto, and the slope of the upper surface of the upper closing surface 2140 may be greater or less than the slope of the lower surface.

Referring back to FIG. 21B, the outer wall 2110 and the inner wall 2130 each having a cylindrical shape may form concentric circles when viewed from the bottom. The outer wall 2110 may surround the inner wall 2130. The outer wall 2110 and the inner wall 2130 may have as much gap as a width 2123 of the lower closing surface formed therebetween due to the lower closing surface 2121. At this time, the heights of the outer wall 2110 and the inner wall 2130 may be equal to each other, and the radius of the outer wall 2110 may be greater than a radius of the inner wall 2130.

Also, the stopper part closing surface 2120 and the upper closing surface 2140 may form concentric circles. When viewed from the bottom, the stopper part closing surface 2120 may have shape surrounding the upper closing surface 2140. That is, a radius of an inner circumferential surface of the stopper part closing surface 2120 may be greater than or equal to a radius of an outer circumferential surface of the upper closing surface 2140. However, the present disclosure is not limited thereto, and the radius of the inner circumferential surface of the stopper part closing surface 2120 may be less than the radius of the outer circumferential surface of the upper closing surface 2140. The stopper part closing surface 2120 may have as much gap as a height 2131 of the inner wall vertically formed due to the upper closing surface 2140 and the inner wall 2130.

The inner body 2020 may include the duct 2150. The duct 2150 may form a passage through which the drug moves. The passage formed by the duct 2150 may pass through the center of the stopper part 2000. The duct 2150 may protrude downward from the upper closing surface 2140 while perpendicular thereto. At this time, a length of the duct 2150 may be shorter than the height of the outer wall 2110 and the inner wall 2130. By the duct 2150 being formed to be shorter than the outer wall 2110 and the inner wall 2130, leakage of the drug to the outside may be minimized. An upper end of the duct 2150 may be coupled to an inner circumferential surface of the upper closing surface 2140. An outer circumferential surface of the duct 2150 may be formed to be inclined.

When a typical hole is formed instead of the duct 2150 that protrudes, a situation in which the drug splashes and partially leaks to the outside without entering the pump module 1930 may occur. Also, when the size of the hole is reduced to allow the drug to entirely enter the pump module 1930, the amount of supplied drug may decrease. However, since the duct 2150 of the present disclosure forms a passage through which the drug moves, the drug inside the drug container 1910 may be discharged toward the pump module 1930 along the duct 2150 regardless of the size of the duct 2150. The upper end of the duct 2150 may be coupled to the inner circumferential surface of the upper closing surface 2140.

A stopper part outer space 2011 and a stopper part inner space 2021 may be formed inside the stopper part 2000 due to the shapes of the outer body 2010 and the inner body 2020 described above.

The stopper part 2000 may include the stopper part outer space 2011 formed by the outer wall 2110, the lower closing surface 2121, and the inner wall 2130. The stopper part outer space 2011 may be a ring-shaped space. An air inlet 2161 of an air hole 2160 may be formed in the stopper part outer space 2011. Impact applied to the stopper part 2000 may be mitigated through the stopper part outer space 2011. Therefore, the stopper part outer space 2011 may prevent main components of the stopper part 2000, such as the duct 2150, from being deformed due to external impact.

The stopper part 2000 may include the stopper part inner space 2021 formed by the inner wall 2130 and the upper closing surface 2140. The stopper part inner space 2021 may be a space surrounded by the stopper part outer space 2011. The stopper part inner space 2021 may be a space for coupling to the pump module 1930. The duct 2150 protruding downward from the upper closing surface 2140 may be formed at an upper side of the stopper part inner space 2021. The stopper part 2000 may minimize leakage of the drug to the outside by the duct 2150 being formed in the stopper part inner space 2021 without protruding to the outside of the stopper part 2000. Specifically, during an attempt to couple the pump module 1930 and the drug container 1910, the amount of the drug flowing down to the outside of the drug container 1910 due to gravity may be minimized.

The stopper part 2000 may include the air hole 2160. Due to the pump module 1930 suctioning the drug inside the drug container 1910 at a high pressure, a situation in which the drug container 1910 is damaged or is detached from the pump module 1930 may occur. The air hole 2160 may form a passage through which air may circulate in the drug container 1910 and may prevent damage to and detachment of the drug container 1910 caused by the high pressure. The air hole 2160 may include the air inlet 2161 and an air outlet 2162. The air hole 2160 may be formed between the outer wall 2110 and the inner wall 2130 and may pass through the stopper part closing surface 2120. That is, the air hole 2160 may be formed in an L-shape. Specifically, the air inlet 2161 of the air hole 2160 may be formed to protrude upward in a vertical direction between the outer wall 2110 and the inner wall 2130. The air outlet 2162 of the air hole 2160 may be formed to pass through at least one of the lower closing surface 2121 and the catching part 2122 in a horizontal direction. Therefore, the air outlet 2162 of the air hole 2160 may be formed in an outer circumferential surface of the stopper part closing surface 2120. By the air hole 2160 being formed in an L-shape instead of a straight shape, when a liquid such as a drug permeates into the air hole 2160, the amount of the liquid leaking to the outside may be reduced. Also, the pump module 1930 may be located below the stopper part 2000. Therefore, when the air hole 2160 is formed in a straight shape, the liquid may permeate into portions other than the container coupling part 2210 of the pump module 1930 and may cause a failure of the pump module 1930. However, since the air hole 2160 is formed in an L-shape, the drug flows outside the pump module 1930, and the liquid may be prevented from permeating into the pump module 1930. Also, when the drug container 1910 is directly coupled to the pump module 1930, when the air hole 2160 is formed in a straight shape, since the air outlet 2162 is blocked by the pump module 1930, it may be difficult to supply the drug. However, when the air hole 2160 is formed in an L-shape, since the air outlet 2162 is not blocked, and air circulation occurs, smooth drug supply may be possible.

In one embodiment of the present disclosure, a drug detector 2170 may be attached to the inside of the inner body 2020. More specifically, the drug detector 2170 may be attached to at least one of the inner circumferential surface of the inner wall 2130 and the lower end of the upper closing surface 2140. The drug detector 2170 may be a substance whose color changes upon contact with a drug. For example, the drug detector 2170 may be litmus paper that reacts with an acid-base of a drug. Alternatively, the drug detector 2170 may be cobalt chloride paper that reacts with water molecules of a drug. However, the drug detector 2170 is not limited thereto and may be any other substance that can detect a drug used in the medical skin treatment apparatus 100. When the drug of the drug container 1910 leaks, the drug may stagnate or flow inside the inner body 2020. Therefore, the user may check leakage of the drug inside the inner body 2020 through a change in the drug detector 2170.

Coupling between the pump module 1930 and the drug container 1910 will be described below.

FIGS. 22A and 22B are views illustrating the drug reservoir according to one embodiment of the present disclosure.

In a state in which the drug container 1910 and the stopper part 2000 are coupled to each other, the drug inside the drug container 1910 may be blocked by at least one of the stopper part closing surface 2120 and the upper closing surface 2140 and may be discharged only through the duct 2150. As described above, the pump module 1930 may suction the drug at a high pressure from the drug container 1910 and discharge the drug. That is, the pump module 1930 may move the drug inside the drug container 1910 to the hand piece 130 through the drug hose 1920.

FIG. 22A illustrates a state in which the drug container 1910 and the stopper part 2000 coupled to each other are coupled to the pump module 1930 through the container coupling part 2210. In one embodiment of the present disclosure, the drug container 1910 and the stopper part 2000 coupled to each other may be coupled to the pump module 1930. The pump module 1930 may include the container coupling part 2210. The container coupling part 2210 may include a structure and a form that couple the drug container 1910 and the stopper part 2000, which are coupled to the pump module 1930, so that the drug inside the drug container 1910 is suctioned into the pump module 1930.

As a coupling structure between the drug container 1910 and the stopper part 2000 which are coupled to the pump module 1930, the container coupling part 2210 may be formed to correspond to the shape of at least one of the drug container 1910, the outer body 2010, the inner body 2020, and the duct 2150. For example, the container coupling part 2210 may include a central protrusion 2211. The central protrusion 2211 having a ring shape may protrude upward past the container coupling part 2210. A diameter of an outer circumferential surface of the central protrusion 2211 may be less than or equal to a diameter of the inner circumferential surface of the inner wall 2130. When the drug container 1910 and the stopper part 2000 coupled to each other are coupled to the container coupling part 2210, the inner circumferential surface of the inner wall 2130 may surround the outer circumferential surface of the central protrusion 2211.

A suction path 2213 may be vertically formed at a center of the central protrusion 2211 along a central axis of the container coupling part 2210. The suction path 2213 may be a passage through which the drug is suctioned. When the drug container 1910 and the stopper part 2000 coupled to each other are coupled to the container coupling part 2210, the duct 2150 of the stopper part 2000 may be in a state in which it is fitted to the suction path 2213 of the container coupling part 2210. Therefore, the drug inside the drug container 1910 may be suctioned into the suction path 2213 of the container coupling part 2210 along the duct 2150 of the stopper part 2000.

Also, the container coupling part 2210 may include an outer boundary protrusion 2212. The outer boundary protrusion 2212 having a ring shape may protrude upward past the container coupling part 2210. A diameter of an inner circumferential surface of the outer boundary protrusion 2212 may be greater than or equal to a diameter of the outer circumferential surface of the outer wall 2110. When the drug container 1910 and the stopper part 2000 coupled to each other are coupled to the container coupling part 2210, the inner circumferential surface of the outer boundary protrusion 2212 may surround the outer circumferential surface of the outer wall 2110. Due to the structure of the container coupling part 2210, the drug container 1910 may be stably fixed to the pump module 1930.

In another embodiment of the present disclosure, the drug container 1910 and the stopper part 2000 coupled to each other may be coupled to the pump module 1930 using the drug hose 1920. FIG. 22B illustrates a state in which the drug container 1910 and the stopper part 2000 coupled to each other are coupled to the pump module 1930 through the drug hose 1920. The outer circumferential surface of the duct 2150 may include a slope that is tilted relative to an axis passing through a center of the upper closing surface 2140. One end of the drug hose 1920 may be fitted from below to surround the outer circumferential surface of the duct 2150. The drug hose 1920 may be coupled to the suction path 2213 of the pump module 1930. Alternatively, the drug hose 1920 may substitute for the role of the suction path 2213 of the pump module 1930. Therefore, the drug discharged through the duct 2150 may be supplied to the pump module 1930 through the drug hose 1920.

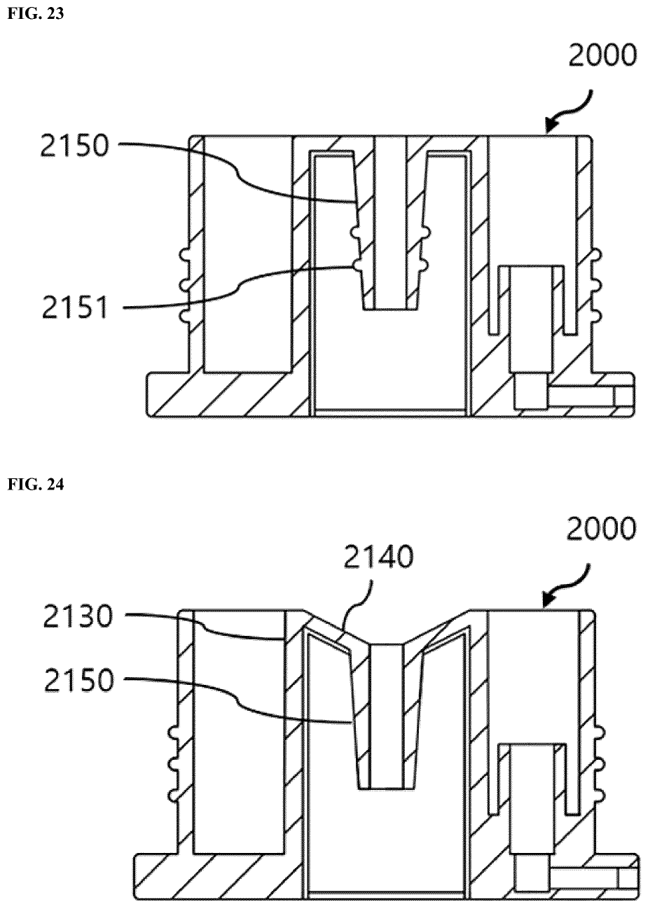

When the drug container 1910 is coupled to the pump module 1930 through the drug hose 1920, the duct 2150 may include at least one duct protrusion. FIG. 23 will be referred to for a moment for description of the duct protrusion.

FIG. 23 is a view illustrating a cross-section of the stopper part according to one embodiment of the present disclosure. A duct protrusion 2151 may be formed to be convex along the outer circumferential surface of the duct 2150. The duct protrusion 2151 may extend in a radial direction of the duct 2150. Due to the convex shape of the duct protrusion 2151, the duct 2150 and the drug hose 1920 may tightly come into close contact with each other. By the duct 2150 and the drug hose 1920 coming into close contact with each other, the drug may be prevented from leaking between the duct 2150 and the drug hose 1920.

The duct protrusion 2151 may be formed of a material that is different from a material of the duct 2150. For example, the duct protrusion 2151 may be formed of a synthetic material with good elasticity and strong friction, such as silicone or rubber, and may increase adhesion between the duct protrusion 2151 and the drug hose 1920. However, the present disclosure is not limited thereto, and the duct protrusion 2151 may be formed of the same material as the duct 2150.

Referring back to FIGS. 22A and 22B, the stopper part 2000 may include an air hose 2200. The air hose 2200 may be formed of an elastic material. One end of the air hose 2200 may be fixed from the top to bottom of the air inlet 2161 of the air hole 2160. Therefore, an inner circumferential surface of the air inlet 2161 and an outer circumferential surface of the air hose 2200 may come into contact with each other, and the air hose 2200 may be fixed to the air inlet 2161 due to friction. The air hose 2200 may extend at a predetermined hose length. The other end of the air hose 2200 may come into contact with at least one of a ceiling surface and a wall surface of the drug container 1910. The predetermined hose length is a length at which the other end of the air hose 2200 is able to come into contact with at least one of the ceiling surface and the wall surface of the drug container 1910 and may be a length at which the other end of the air hose 2200 is able to be located on an air layer inside the drug container 1910 that is formed as the drug is consumed. The predetermined hose length may vary according to the size of the drug container 1910. When the pump module 1930 suctions the drug inside the drug container 1910 at a high pressure, a relatively low air pressure may be formed in the drug container 1910, and the drug container 1910 may be crushed as a result. Also, when the drug inside the drug container 1910 is supplied to pump module 1930 due to gravity, a relatively low air pressure may be formed in the drug container 1910 over time, and the drug may not be supplied any more. However, since the air layer inside the drug container 1910 is connected to the outside through the air hose 2200 and the air hole 2160, the pressure inside the drug container 1910 can be maintained. Therefore, the drug can be smoothly supplied to the pump module 1930.

A skin treatment apparatus of the present disclosure includes a stopper part coupled to a drug container. The stopper part prevents leakage of a drug inside the drug container. Also, the stopper part has an air hole formed therein to prevent the drug container from being crushed by a high pressure.

The advantageous effects that can be obtained by the present disclosure are not limited to the advantageous effect mentioned above, and other unmentioned advantageous effects may be clearly understood by those of ordinary skill in the art to which the present disclosure pertains from the description above.

Various embodiments have been described above. Those of ordinary skill in the art to which the present invention pertains should understand that the present invention may be implemented in modified forms within the scope not departing from essential characteristics of the present invention. Therefore, the embodiments disclosed herein should be considered in an illustrative aspect instead of a limiting aspect. The scope of the present invention is shown by the claims rather than the above description, and all differences present within the scope equivalent to the claims should be construed as being included in the present invention.

Meanwhile, the above-described embodiments of the present invention may be written by a program that may be executed by a computer, and may be implemented by a universal digital computer operating the program using computer-readable recording media. The computer-readable recording media include storage media such as magnetic storage media (for example, a ROM, a floppy disk, a hard disk, and the like) and optical readable media (for example, a compact disc (CD)-ROM, a digital versatile disc (DVD), and the like).

What is claimed is:

1. A medical skin treatment apparatus comprising:
a main body including a controller controlling an operation of the medical skin treatment apparatus and a power supply for supplying power to the medical skin treatment apparatus;
a drug reservoir storing a drug to be injected into the skin of a patient;
a hand piece receiving power from the power supply of the main body, controlled by the controller, injecting the drug into the skin of the patient, and improving the skin; and
a drug hose coupling the drug reservoir and the hand piece to each other and conveying the drug,
wherein the drug reservoir includes a drug container storing the drug and a stopper part inserted into an opening of the drug container to prevent leakage of the drug and having a duct through which the drug hose passes formed therein,
the stopper part includes an outer body having at least a portion coming into contact with an inner circumferential surface of the opening of the drug container and an inner body,
the outer body includes an outer wall and a stopper part closing surface having a ring shape and integrally formed with the outer wall,
the outer wall has a cylindrical shape that has an open upper side and vertically extends,
the stopper part closing surface includes a lower closing surface and a catching part,
the lower closing surface is formed in a ring shape that is perpendicular to the outer wall, and an outer circumferential surface of the lower closing surface is coupled to a lower end of the outer wall,
the catching part extends in a radial direction of the lower closing surface and is formed to be larger than a radius of the outer wall to prevent the outer body from being completely inserted into the drug container,
the inner body includes an inner wall, an upper closing surface that are integrally formed with the inner wall, and the duct,
the inner wall has a cylindrical shape that is coupled to the lower closing surface while perpendicular thereto, has an open lower side, and vertically extends, and a lower end of the inner wall is coupled to an inner circumferential surface of the lower closing surface,
the upper closing surface is formed in a ring shape that is perpendicular to the inner wall, and an outer circumferential surface of the upper closing surface is coupled to an upper end of the inner wall,
the duct is formed to protrude downward while perpendicular to the upper closing surface and discharge the drug inside the drug container to the outside, and
an upper end of the duct is coupled to an inner circumferential surface of the upper closing surface.

2. The medical skin treatment apparatus of claim 1, wherein:
the outer wall and the inner wall each having a cylindrical shape form concentric circles, and the radius of the outer wall is greater than a radius of the inner wall; and
the stopper part closing surface and the upper closing surface form concentric circles, and a radius of the stopper part closing surface is greater than a radius of the upper closing surface.

3. The medical skin treatment apparatus of claim 2, wherein:
an outer circumferential surface of the duct includes a slope that is tilted relative to an axis passing through a center of the upper closing surface;
the drug hose is fitted from below to surround the duct;
in a state in which the drug container and the stopper part are coupled to each other, the drug is blocked by at least one of the lower closing surface and the upper closing surface and is discharged only through the duct; and
the drug discharged through the duct is supplied to the hand piece through the drug hose.

4. The medical skin treatment apparatus of claim 3, wherein the stopper part further includes:
an air hole that includes an air inlet and an air outlet, is formed between the outer wall and the inner wall, passes through the stopper part closing surface, and is formed in an L-shape; and
an air hose that is formed with an elastic material and has one end fixed from above to the air inlet of the air hole and the other end extending at a predetermined hose length to come into contact with at least one of a ceiling surface and a wall surface of the drug container.

5. The medical skin treatment apparatus of claim 4, wherein:
the air inlet of the air hole is formed to protrude in a vertical direction between the outer wall and the inner wall; and
the air outlet of the air hole is formed to pass through at least one of the lower closing surface and the catching part in a horizontal direction.

6. The medical skin treatment apparatus of claim 5, wherein:
the outer body has at least one stopper protrusion formed along an outer circumferential surface of the outer wall to allow the stopper part and the drug container to tightly come into close contact with each other; and
the stopper protrusion extends in a radial direction of the outer wall and prevents the drug from leaking between the outer wall and the drug container.

7. The medical skin treatment apparatus of claim 6, wherein:
the duct has at least one duct protrusion formed along the outer circumferential surface of the duct to allow the stopper part and the drug hose to tightly come into close contact with each other; and
the duct protrusion extends in a radial direction of the duct and prevents the drug from leaking between the duct and the drug hose.

8. The medical skin treatment apparatus of claim 7, wherein a drug detector is attached to at least one of an inner circumferential surface of the inner wall and a lower end of the upper closing surface, which are at an inside of the inner body, to check leakage of the drug.
